(19) United States
(12) Patent Application Publication
(10) Pub. No.: US 2009/0093023 A1
(43) Pub. Date: Apr. 9, 2009

(54) Elements for improved expression of bovine somatotropin

(76) Inventors: Gregg Bogosian, Clarkson Valley, MO (US); Julia P. Frantz, Glendale, MO (US); Hong Q. Smith, St. Louis, MO (US)

Correspondence Address: HOWREY LLP, C/O IP DOCKETING DEPARTMENT, 2941 FAIRVIEW PARK DRIVE SUITE 200, FALLS CHURCH, VA, 22042, US

(21) Appl. No.: 12/009,332

(22) Filed: Jan. 17, 2008

Related U.S. Application Data
(60) Provisional application No. 60/881,336, filed on Jan. 19, 2007.

(51) Int. Cl. C12P 21/00; C07H 21/00; C12N 15/63; C12N 1/00; C12N 1/21

(52) U.S. Cl. 435/69.4; 536/23.1; 435/320.1; 435/243; 435/252.33

(57) ABSTRACT

The invention allows improved expression of heterologous polypeptides such as bovine somatotropin (bST). Novel compositions and methods are provided for production of bST from a native bST cDNA in transformed host cells such as E. coli. In particular, DNA segments comprising novel ribosome binding sites and adjacent sequences are provided, which in conjunction with a promoter sequence direct high level expression of bST from its native cDNA. The invention also provides expression constructs comprising the ribosome binding sites and methods for the use of such constructs.

This application claims the benefit of priority to U.S. Provisional Application No. 60/881,336, filed Jan. 19, 2007, which is herein incorporated by reference in its entirety.

### BACKGROUND OF THE INVENTION

1. Field of the Invention

The present invention relates generally to methods and compositions for improving expression levels of heterologous polypeptides in transformed host cells. More specifically, it relates to improved expression of bovine somatotropin (“bST”) protein encoded by the native bST gene complementary DNA (cDNA), by use of novel expression elements.

2. Description of Related Art

With the advent of gene cloning techniques in the early to mid 1970's, including the cloning of gene cDNAs, molecular biologists in many laboratories began cloning the cDNAs for mammalian growth hormones and related hormones. By 1979, the methods for cloning growth hormone cDNA were routine and had been described in many papers (Seeburg et al., 1977a; Seeburg et al., 1977b; Shine et al., 1977; Harpold et al., 1978; Goodman and MacDonald, 1979; Gubbins et al., 1979; Martial et al., 1979; Roskam and Rougeon, 1979; Roberts et al., 1979; and Fiddes and Goodman, 1979). The early successes with the cloning of mammalian growth hormone genes, along with detailed discussions of the methods employed, were the subject of several review articles written in 1979 (Efstratiadis and Villa-Komaroff, 1979; Taylor, 1979; Wu, 1979; and Miller, 1979).

The cloning of mammalian growth hormone genes was also the subject of several patent applications filed in the late 1970's (Rutter, South African Patent Application 782805; French Patent Application 7815596; Rutter, British Patent 1565190; Itakura, U.S. Pat. Nos. 4,356,270, 4,704,362, 5,221,619; Itakura, UK Patent Application 2007676; Goodman, U.S. Pat. No. 4,363,877; Rutter, European Patent 0012494 B1; Baxter, European Patent 0020147 B2; and Goeddel, U.S. Pat. No. 4,342,832). Several of these papers, patents, and patent applications indicate that the methods described for the cloning of the cDNA genes for mammalian growth hormones would be applicable to the cloning of the cDNA gene for bovine growth hormone (bGH), also known as bovine somatotropin (bST).

Prior to the cloning of the bST cDNA gene, the complete amino acid sequence of the mature bST protein (SEQ ID NO:29 and SEQ ID NO:110) had already been established and independently confirmed (Wallis, 1973; Graf and Li, 1974; Wallis and Davies, 1976; and Dayhoff, 1976). The stage was thus set for the cloning of the bST cDNA gene. Several laboratories soon reported the successful cloning of the bST cDNA gene (Israeli Patent Application 59690; Aviv, French patent application 19815731; Aviv, UK Patent GB 2073245 B; Aviv, U.S. Pat. No. 6,229,003 B1; Miller et al., 1980a; Miller et al., 1980b; Miller, European Patent 0047600 B1; Miller, U.S. Pat. No. 6,692,941 B1; Keshet et al., 1981; Woychik et al., 1982; Rottman, European Patent Application 0067026 A1; de Boer, European Patent 0075444 B1; de Boer, European Patent 0278121 B1; de Boer, U.S. Pat. Nos. 4,880,910, 5,254,463, 5,260,201, and 5,489,529; Buell, U.S. Pat. No. 4,693,973; Glassman, European Patent Application 0111814 A2; Franke, European Patent 0147178 B1; Seeburg et al., 1983; and Rubtsov et al., 1985).

The nucleotide sequences of the various bST cDNA genes that were obtained by the researchers mentioned in the previous paragraph were all in agreement with the confirmed amino acid sequence of the mature bST protein. With one notable exception, the nucleotide sequences of the bST cDNA genes obtained by these workers were also in agreement with each other. The exception was the bST cDNA sequence described by Miller et al. (1980a: 1980b; Miller, European Patent 0047600 B1; and Miller, U.S. Pat. No. 6,692,941 B1). The bST cDNA gene sequence of Miller and co-workers has several differences with the other published sequences, differences that were later found to be errors that had occurred in the sequencing of the bST cDNA gene by Miller and co-workers. These errors were confirmed by sequencing the plasmid pBP348 containing the cloned bST cDNA gene of Miller et al., available from the American Type Culture Collection, Manassas, Va., as accession number ATCC 31686. This sequencing showed that the differences between the bST cDNA sequence reported by Miller et al. and the bST cDNA sequences reported by others were due to errors by Miller and co-workers.

The correct nucleotide sequences of the four known variant coding sequences (discussed below) for the portion of the native bST cDNA gene encoding the mature bST protein are given here as SEQ ID NO:27, SEQ ID NO:107, SEQ ID NO:108 and SEQ ID NO:109.

Bovine somatotropin protein isolated from mixtures of bovine pituitary glands consists of four different variant protein molecules. Heterogeneity at the amino-terminus of bST was first recognized by Reid (1951) and Li and Ash (1953), who reported both alanine and phenylalanine as the amino-terminal amino acid. Work by others established that the proteins differed by the presence or the absence of an extra amino-terminal alanine amino acid (Fellows and Rogol, 1969; Wallis, 1969; Pena et al., 1969; Pena et al., 1970; Fellows et al., 1971; Seavey et al., 1971; Santome et al., 1971; Fellows et al., 1972; Wallis, 1973; and Santome et al., 1973; Wallis and Davies, 1976; Wood et al., 1989). The two forms were found to occur in a 1:1 ratio. It was found that every individual pituitary gland contains both forms in the 1:1 ratio, ruling out allelic polymorphism as the origin of the amino-terminal heterogeneity.

Amino acid sequencing of the bST translation product of bovine pituitary mRNA in a wheat germ cell-free system revealed that the pre-bST protein was synthesized with a 26 or 27 amino acid presequence (Lingappa et al., 1977). When microsomal membranes from canine pancreas or bovine pituitary were added, the pre-bST protein was converted into the mature bST protein. The processed bST protein exhibited an Ala-Phe/Phe amino-terminal heterogeneity. This observation led these workers to conclude that the cleavage site in nascent pregrowth hormone is sufficiently ambiguous as to result in random cleavage either before or after alanine. Thus, variable processing of the bST protein precursor molecule is the origin of the observed amino-terminal heterogeneity.

As various laboratories sequenced the entire bST protein, they reported that both leucine and valine occur at position 126 in the amino acid chain (Fellows and Rogol, 1969; Seavey et al., 1971; Santome et al., 1971; Fellows et al., 1972; Wallis, 1973; and Santome et al., 1973; Wallis and Davies, 1976; Wood et al., 1989). The ratio of leucine to valine at position 126 was about 2:1. With bST protein prepared from individual pituitary glands, the amino acid(s) at position 126 could be all leucine, all valine, or a mixture of both (Seavey et al., 1971). This demonstrated that the variation at position 126 is due to allelic polymorphism. Combined with the variable processing of the bST precursor molecule, the four different variant mature bST protein molecules obtained from mixtures of bovine pituitary glands are thus Ala-Phe-(Leu-126)-bST (SEQ ID NO:29), Ala-Phe-(Val-126)-bST (SEQ ID NO:110), and these two sequences without the first alanine amino acid, namely Phe-(Leu-126)-bST and Phe-(Val-126)-bST, respectively.

Sequencing of the bST gene from a single cDNA clone could not confirm the existence of the allelic polymorphism at position 126 since only one cDNA clone was sequenced (Miller et al., 1980b); the bST cDNA sequence reported by these workers had a leucine CTG codon at position 126. Similarly, the sequencing of two different single clones of the genomic bST gene (with introns and exons) by two different laboratories also could not confirm the allelic polymorphism; these two clones also had leucine CTG codons at position 126 (Woychik et al., 1982; Gordon et al., 1983). The sequencing of another single bST cDNA clone also had a leucine CTG codon at position 126, again failing to confirm the existence of the allelic polymorphism (Seeburg et al., 1983). Two other bST cDNA clones, namely the plasmid pLG23 (Rottman, European Patent Application 0067026 A1) and the plasmid D4 (Aviv, U.S. Pat. No. 6,229,003 B1) were recently obtained (plasmid pLG23 is available from the Northern Regional Research Laboratory, Peoria, Ill., under accession number NRRL B-12436, and plasmid D4 is available from the American Type Culture Collection, Manassas, Va., under accession number ATCC 31826) and their nucleotide sequences determined. Again, both of these bST cDNA clones also had a leucine CTG codon at position 126.

Apparently the first time a bST cDNA clone with the valine 126 codon was obtained and sequenced was in a bST cDNA clone made from bovine pituitary tissue described in a United States patent filed in 1983 (Buell, U.S. Pat. No. 4,693,973). Buell obtained a bST cDNA clone designated pBGH108, sequenced the insert and found valine GTG at position 126. This work established that the valine GTG codon occurred at position 126 in variants with this allele. In 1993, Lucy et al. published a paper that confirmed the allelic variation at codon 126 was caused by a nucleotide change of CTG to GTG in the bST gene.

Another allelic variant within the native bST sequence was also identified at codon 188 (Seeburg et al., 1983; U.S. Pat. Nos. 4,880,910; 5,254,463; 5,489,529; 5,260,201; and European Patents 0075444 B and 0278121 B1). This codon was found to be either TGT or TGC, both coding for Cys. Thus, there are four known variant coding sequences for the portion of the native bST cDNA gene encoding the mature bST protein: 1. Leu-126 CTG, Cys-188 TGT (SEQ ID NO:27); 2. Leu-126 CTG, Cys-188 TGC (SEQ ID NO:107); 3. Val-126 GTG, Cys-188 TGT (SEQ ID NO:108); and 4. Val-126 GTG, Cys-188 TGC (SEQ ID NO:109).

The numbering system for the codons and corresponding amino acids of bST is based on the convention of designating the first phenylalanine as amino acid +1 (Wood et al., 1989). In this numbering system, the preceding alanine amino acid, present in about 50% of the mature bST molecules obtained from bovine pituitary tissue, is designated amino acid −1. In addition to the mature bST coding sequence, a cloned cDNA copy of the bST gene would also include codons for the 26 amino acid presequence (also referred to as the leader peptide) and flanking nucleotide sequences derived from the bovine pituitary mRNA molecule used to generate the cDNA copy.

The main objective for cloning cDNA copies of genes for growth hormones and other proteins is to use such genes to construct recombinant expression plasmids that can be employed to produce large quantities of the proteins in recombinant strains of Escherichia coli. For commercial purposes, such as the use of human growth hormone in the treatment of human pituitary dwarfism, kilogram quantities of the recombinant human growth hormone are required each year. Even more daunting is the challenge of commercially producing the tens of metric tons (a metric ton is one thousand kilograms) of mature bST protein needed each year for the use of bST in the dairy industry. This need necessitated the development of expression systems that yield at least several grams of bST protein per liter of fermentation culture in order to provide a commercially feasible manufacturing process (Kane and Bogosian, 1987; Calcott et al., 1988; Kane et al., 1991).

Having a cDNA clone of a gene does not by itself yield the desired protein. The cDNA gene must be inserted into an effective “expression vector”. At a minimum, an expression vector must have a promoter (the site that will be recognized by bacterial RNA polymerase) so that transcription of the cDNA can be initiated. Furthermore, the cDNA must contain a bacterial ribosome-binding site upstream from the first codon, so that the transcribed RNA can be translated on bacterial ribosomes. However, use of proven promoters and ribosome binding sites, i.e. those that had been effective in expressing other proteins, were consistently unable to express the cDNA for mature bST. These prior efforts at expressing bST protein are discussed below.

The cloning of a cDNA copy of a growth hormone gene was only the first of many steps to achieving high level expression of mature growth hormone protein in a recombinant E. coli strain (Calcott et al., 1988). The cDNA copy of the bST gene is not ready to use for expression of the mature bST protein, encumbered as it is with the codons for the presequence and other flanking nucleotide sequences. These flanking sequences must be precisely removed, and the codons encoding the mature bST protein (SEQ ID NOs:27, 107, 108, or 109) must be linked to suitable gene expression elements that are functional in E. coli strains. These gene expression elements include proper placement of an ATG start codon in front of the bST codons, to yield a structural gene encoding either Met-Ala-Phe-bST or Met-Phe-bST (depending on whether the objective is to produce mature bST protein with or without the first alanine amino acid at position-1). The first methionine amino acid is retained on the bST protein if the next amino acid is phenylalanine, but this initial methionine is not retained on the bST protein if the next amino acid is alanine (Calcott et al., 1988; Warren et al., 1996).

Early efforts reported in the literature at expressing the mature bST protein fell into three categories: (1) attempts at expressing the unmodified portion of the bST cDNA gene encoding the naturally occurring mature bST molecule, using proven standard available expression systems; (2) attempts at expressing a modified bST gene by adding or deleting codons or introducing silent changes in the gene encoding the amino-terminal portion of the protein; and (3), attempts at expressing the unmodified bST cDNA sequence using a unique expression system with a two-cistron messenger RNA, and modifications derived from that two-cistron system.

1. Attempts to express the unmodified bST cDNA gene encoding mature bST protein, using proven expression systems.

Almost all of the early research groups that had obtained a cDNA clone of the bST gene went on to attempt expression of the mature bST protein in various standard (one ribosome binding site and one cistron) and proven expression systems available at the time of their investigations. The group of Miller and co-workers was an exception. Miller et al. obtained a cDNA clone of the bST gene, albeit with sequencing errors, but did not go on to attempt expression of the mature bST protein (Miller et al., 1980a; Miller et al., 1980b; Miller, European Patent 0047600 B1; and Miller, U.S. Pat. No. 6,692,941 B1). However, other research groups that had a cDNA clone of the bST gene proceeded to construct various standard expression systems designed to produce the mature bST protein. They assumed that expression of the mature bST protein in a recombinant E. coli strain could be achieved with the construction of a standard expression system, consisting of a promoter, single ribosome binding site, ATG start codon, and the codons for the mature bST protein obtained from a cDNA copy of the bST gene. Indeed, cDNA copies of other growth hormone genes were found to be readily expressed in such systems, including reports of high level expression of mature growth hormones from cDNA genes for human growth hormone (Mayne et al., 1984), chicken growth hormone (Souza et al., 1984), and salmon growth hormone (Sekine et al., 1985). However, the workers attempting to express mature bST protein ran into an unexpected obstacle. They all found that the bST cDNA gene did not express mature bST protein at detectable levels, or only expressed the bST protein very poorly, in various standard E. coli gene expression systems.

de Boer et al. (U.S. Pat. Nos. 4,880,910, 5,254,463, and 5,260,201) reported no bST production, or poor production, using the culture containing natural bST cDNA gene. Buell (U.S. Pat. No. 4,693,973) observed using bST cDNA clones that the expression yields of bST protein in various hosts were too low to provide economically useful or commercial quantities of bST protein. They placed the bST cDNA gene under the control of various standard expression systems, and obtained only up to 100 bST molecules per cell. They concluded that mere placement of an ATG start codon at the beginning of the DNA sequence coding for mature bovine growth hormone did not permit the synthesis of useful amounts of bST protein in E. coli. Glassman et al. (European Patent Application 0111814 A2) observed that expression of bST cDNA sequences was very low (less than 0.01% of the total protein expressed by the cell) in a number of proven standard expression vector constructions. The same observations were reported by these workers in Krzyzek et al. (1984). When the bST cDNA gene was placed in yet another proven standard expression system by these workers, they again found that production of bST protein was extremely low (George et al., 1985).

Schoner et al. (1984) reported that in their proven standard expression systems with a bST cDNA gene, expression of bST protein was very low or undetectable. They suggested that secondary structures in the mRNA might explain failures to express the bST cDNA gene at high levels, and later observed that no detectable amounts of bST protein were produced from another proven standard expression plasmid with a bST cDNA gene (Schoner et al., 1986). Schoner et al., 1987 also observed that the bST cDNA gene did not express at high levels in proven standard expression systems that had been used to overproduce other eukaryotic proteins. [Note: Schoner and coworkers at first reported that a standard expression system designed to express mature bST from a bST cDNA gene, designated plasmid pCZ108, produced bST at about 1.7% of the total cell protein (Schoner et al., 1984). However, in a subsequent publication (Hsiung and McKellar, 1987), they reported that pCZ108 did not employ an unmodified bST cDNA gene, but rather a bST gene with a silent alteration of the codon for leucine +6 from the cDNA codon of TTG to the cognate, non-cDNA leucine codon CTG (see discussion of expression using silent changes, in Section 2 below)].

Szoka et al., 1986 reported that for bST, the use of direct expression vectors with the native bST cDNA sequence resulted in very poor expression in E. coli. Olson et al., 1987 reported that an expression system employing an unaltered bST cDNA gene did not produce detectable amounts of bST. They also reported that the native (unmodified) bST cDNA is poorly expressed in E. coli (Watson and Olson, 1987; Tomich and Olson, 1989; Tomich et al., WO 89/07141), and reported that their bST cDNA expression plasmid did not produce detectable levels of bST protein (Watson and Olson, 1990). Choi and Lee (1996) reported a lack of bST expression regardless of promoter strength, optimal Shine-Dalgarno sequence, host strains, and culture conditions.

These workers recognized that the bST cDNA sequence had a structural feature in the beginning of the bST gene that prevented or significantly interfered with expression in E. coli in the standard expression systems they had investigated.

In fact, the consensus of these workers regarding proven standard expression systems for expressing bST cDNA in E. coli was that such an approach was not successful. For example, Schoner et al., in reporting negative results, stated that “no detectable amounts of Met-Ala-bGH were produced (as expected)” (emphasis added) by cultures harboring a proven standard expression plasmid with a bST cDNA gene (Schoner et al., 1986). They also observed that “The bGH gene (with its native codons) does not express at high levels in conventional vectors that have been used to overproduce other eukaryotic proteins” (Schoner et al., 1987).

Watson and Olson, 1990 reported that with their bST cDNA expression plasmid pSK102, “like other pBR322-based vectors containing this version of the bGH cDNA, induced cells containing pSK102 did not produce detectable levels of bGH”. Choi and Lee 1996 stated that “expression from the unmodified cDNA coding for bGH have been proven to be difficult by the usual vector systems. The lack of bGH expression is likely due to some properties in the DNA sequence itself.” In 1999, Choi and Lee in effect summarized the experience of those working in the art when they observed that “The expression of unmodified cDNA coding for bGH has proven difficult regardless of promoter strength, optimal Shine-Dalgarno sequence, host strains, and culture conditions, indicating that the bGH coding sequence has some inherent characteristics preventing its high level expression in E. coli” (Choi and Lee, 1999).

2. Expressing a modified DNA sequence by adding or deleting codons or introducing silent changes into the codons in the region of the bST gene encoding the amino-terminal portion of the bST protein.

Researchers sought and found alternatives to overcome this obstacle to expressing bST from cDNA using standard expression systems. One successful approach involved adding, deleting, or altering the codons in the beginning of the bST cDNA gene in order to remove the hypothesized problematic structural feature from the beginning of the bST gene. All of these approaches could result in high-level expression of the bST protein, and a number of patents were issued (e.g. U.S. Pat. Nos. 6,828,124, 4,880,910, 5,254,463, 5,260,201, 5,489,529, 6,229,003, 5,059,529, 4,693,973, 4,673,641, 4,935,370, 5,395,761, 5,240,837, 5,366,876 and 5,955,297, and European Patent 0095361; see also Calcott et al., 1988; Kane et al., 1991).

It should be noted, however, that when the solutions involved adding various codons to the beginning of the bST gene, or deleting various codons from the beginning of the bST gene, the result was a system that did not express mature bST protein, but rather would produce variant proteins with different amino acid sequences than mature bST protein.

However, the approach of altering the codons in the beginning of the bST cDNA sequence was also found to succeed. If certain nucleotide changes were made to the bST cDNA gene in the beginning of the bST coding region, then as long as the alterations were “silent”, that is, as long as they changed a cDNA codon to a cognate codon encoding the same amino acid, the bST protein produced would have the same amino acid sequence as mature bST protein. This approach was capable of producing mature bST protein at a high enough yield to be suitable for commercial purposes. These silent alteration approaches were those taken by de Boer et al. (U.S. Pat. Nos. 4,880,910, 5,254,463, 5,260,201, and 5,489,529), and by Bogosian et al. (U.S. Pat. No. 6,828,124), to achieve commercial level expression of mature bST protein.

3. Expressing the unmodified cDNA sequence using an expression system using two cistrons and modifications derived from that system.

Schoner et al. developed a non-standard expression system, that they termed a “synthetic two-cistron mRNA”, comprising two ribosome sites and an additional cistron that was able to express mature bST from a bST cDNA gene at levels up to 25% of the total cell protein. These workers later made modifications to the coding region of the first cistron resulting in an expression system comprising two ribosome binding sites but no longer with an intact first cistron (Schoner, European Patent Application 0154539 A2; Schoner et al., 1984; Schoner et al., 1986; and Schoner et al., 1987).

Thus, it appears that no worker has succeeded in achieving high level expression (greater than 300 milligrams per liter in a culture achieving the high cell densities typical of growth in a fermentation vessel) of an unmodified cDNA gene for bST using a standard expression system with a single ribosome binding site. This gave rise to the question of whether single ribosome binding site expression systems could be developed for expressing bST cDNA at significant levels.

Consequently, it is an object of the present invention to provide single ribosome binding site expression systems effective for expressing bST cDNA. It is another object of this invention to provide novel and effective ribosome binding sites suitable for use generally in expression systems. Further objects of the invention include methods and processes using these expression systems and ribosome binding sites. The accomplishment of these objectives will be understood and appreciated by the skilled artison by referring the following description of the invention and the claims.

### BRIEF DESCRIPTION OF THE FIGURES

FIG. 1: Restriction map of bST expression cassette

FIG. 2: Ribosome binding sites and corresponding bST expression levels

### SUMMARY OF THE INVENTION

The present invention provides compositions and methods for expressing heterologous polypeptides in transformed host cells. In certain embodiments, the heterologous polypeptide is bovine somatotropin (bST). In one embodiment, the present invention provides an isolated nucleic acid sequence comprising a ribosome binding site sequence that is at least about 80% similar to SEQ ID NO:106. In other embodiments, the isolated nucleic acid sequence comprising a ribosome binding site sequence is at least about 84%, at least about 88%, at least about 92%, or at least about 96% identical to SEQ ID NO:106. In yet another embodiment, the ribosome binding site comprises SEQ ID NO:106.

The invention further provides recombinant constructs comprising such ribosome binding site sequences, operably linked to a functional promoter and a sequence corresponding to any one of the four native cDNA sequences coding for bST (SEQ ID NO:27, SEQ ID NO:107, SEQ ID NO:108, or SEQ ID NO:109). In one embodiment, the cDNA sequence encoding bST comprises a native cDNA sequence that encodes the polypeptide sequence of SEQ ID NO:29, SEQ ID NO:110, SEQ ID NO:112 or SEQ ID NO:113. In another embodiment, the promoter comprises the nucleotide sequence of SEQ ID NO:26. The invention also provides recombinant host cells comprising such recombinant constructs, for example prokaryotic host cells (e.g. E. coli host cells) comprising such a construct.

Other recombinant constructs comprising a ribosome binding site sequence in operable linkage to a native bST cDNA sequence constitute further embodiments of the invention. In one of these embodiments, the ribosome binding site is characterized as containing the subsequence DDAGGDD. The central G of this subsequence is located 10 to 13 nucleotides 5′ of (upstream) the bST cDNA start codon; the 6 to 9 nucleotides between the subsequence and the bST start codon comprise at least four adenine and/or thymine nucleotides. Another ribosome binding site that can be used in bST recombinant constructs is at least 80% identical to SEQ ID NO:106. The last eight nucleotides at the 3′-end of this ribosome binding site sequence contain at least seven adenine and/or thymine nucleotides. bST recombinant constructs having either of these ribosome binding site sequences may further contain a promoter, such as the sequence of SEQ ID NO: 26, that functions in a host cell (e.g. E. coli). The bST cDNA sequence of these constructs may be that of SEQ ID NO:29, SEQ ID NO:110, SEQ ID NO:112, SEQ ID NO:112 or SEQ ID NO:113, for example.

The invention further provides methods for producing a heterologous polypeptide in transformed host cells, comprising: (a) obtaining a host cell with a recombinant construct of the present invention; and (b) culturing the host cell under conditions that induce the expression of the coding sequence. In one embodiment, the heterologous polypeptide is bST. In another embodiment, the host cell is a prokaryotic cell, such as E. coli.

### DETAILED DESCRIPTION OF THE INVENTION

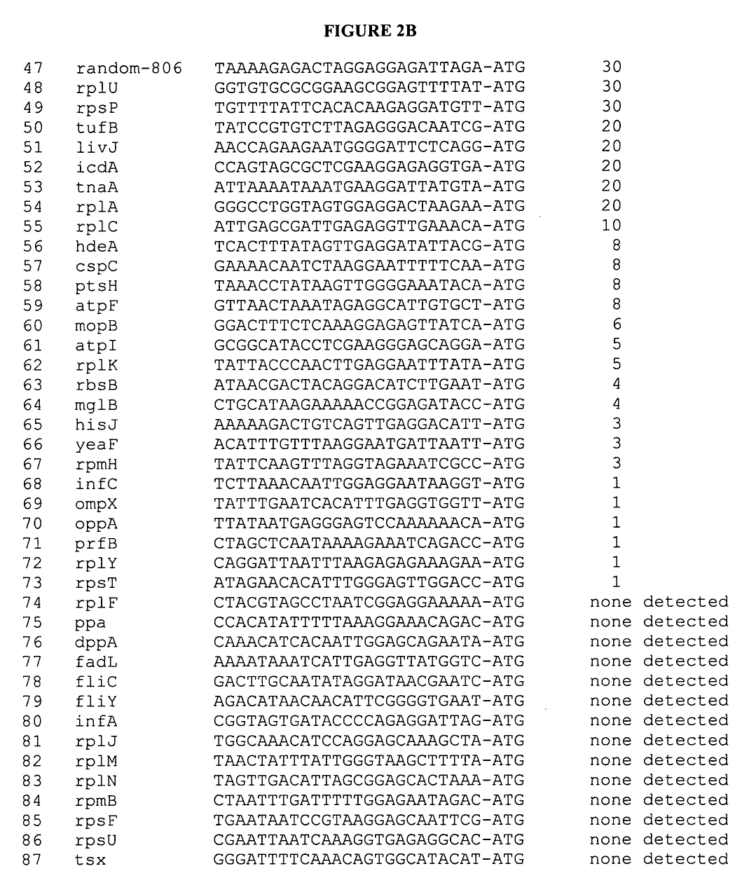

Described herein is a set of novel ribosome binding sites and flanking sequences (collectively termed “RBS” herein) that are of use in expressing bST, and which enable expression of bST at high levels even from native bST cDNA using conventional fermentation and induction conditions (e.g. Bogosian et al. 1989). This set is exemplified by the RBS sequences listed as SEQ ID NO:1-25 and as shown in FIG. 2. An expression vector comprising a functional bacterial promoter operably linked to the RBS of any one of SEQ ID NOs:1-25, and a bST structural gene coded by the native cDNA and ending with a translation stop codon for the structural gene, allows for bST protein expression of greater than 300 milligrams per liter. A bST cDNA sequence is found at SEQ ID NO:27. This sequence as well as its allelic variants at sequences corresponding to codons 126 and codon 188 (e.g. SEQ ID NOs:107-109) each comprise native bST cDNA sequences. As used herein, the term “native bST cDNA” refers to bST cDNA that has not been subject to any form of intentional mutation, such as by site-directed mutagenesis.

An RBS is a sequence of nucleotides near the 5′ end of an mRNA molecule that facilitates the binding of the mRNA to the small ribosomal sub-unit of the ribosome complex. With respect to bacterial mRNA's, this sequence generally contains a Shine-Delgamo sequence domain; however, a Shine-Delgamo domain is not completely necessary for RBS function. Binding of the ribosome to the RBS is a critical step in initiating protein translation from an mRNA transcript. The RBS is usually located just upstream the AUG start codon of a gene.

The present invention allows, for the first time, detectable expression of bST polypeptides using native bST cDNA coding sequences in host cells transformed with an expression vector of the present invention, such as the bacterium E. coli. A promoter functional in the transformed host cell, such as the synthetic promoter designated “cpex-20” (Bogosian et al., U.S. Pat. No. 6,617,130), may be employed. Numerous bacterial promoters are known in the art (e.g. Lisser and Margalit, 1993; Chasov et al., 2002), and various other conventional and novel promoters can be used in these vectors in lieu of cpex-20 to achieve good levels of bST expression. A sequence of the present invention (e.g. SEQ ID NO:1-25) comprising an RBS may be placed downstream of the promoter. Restriction enzyme sites may also be included to facilitate cloning and manipulation of the DNA segments, such as those found in an expression cassette of pXT709 (SEQ ID NO:28). Any of the four cDNA sequences of a native bST gene (SEQ ID NO:27, SEQ ID NO:107, SEQ ID NO:108, or SEQ ID NO:109) can be placed downstream of the RBS, followed by a stop codon and a terminator, examples of which are known, such as the tandem lacUV5 sequence. The translation stop codon may comprise one or more tandem stop codons, and another terminator may also be employed.

A recombinant expression construct or vector comprising the above elements, in which a promoter functional in a transformed host cell, operably linked to an RBS of the present invention, and a native bST cDNA, was prepared. The DNA segments used to prepare the expression construct were synthesized or isolated from an available source, and the identity of the construct verified by DNA sequencing. The resulting plasmid vector, with an expression cassette derived from that of pXT709 (SEQ ID NO:28), with a novel RBS of the present invention and a native bST coding sequence, was then transformed into E. coli K-12 host strain LBB427. Strain LBB427 is nearly identical to the standard wild-type E. coli K-12 strain W3110; the only difference being that strain LBB427 has a mutation in the fhuA gene. This mutation does not affect bST expression in any way. Other E. coli host strains known in the art may also be employed. The nucleotide sequences of the portion of the four native bST cDNA genes encoding the mature bST protein are found at SEQ ID NO:27, SEQ ID NO:107, SEQ ID NO:108, or SEQ ID NO:109, and the two variant corresponding peptide sequences are found at SEQ ID NO:29 or SEQ ID NO:110.

Means for transforming bacteria such as E. coli are well known in the art, and the ability to transfer plasmids into E. coli is an important research tool. Much work has been done to define the parameters involved in bacterial transformation with DNA, with the goals of improving transformation frequency (e.g. Hanahan et al., 1983, 1991; Sambrook et al., 1989). Many factors improve transformation frequency, including genetic factors, and physical and chemical treatments such as heat shock, inclusion of monovalent or divalent cations in the transforming buffer, the addition of DMSO, PEG-8000, hexamine cobalt chloride, treatment with solvents and sulfhydryl reagents, and growth in media containing elevated magnesium levels. Manipulating these parameters has improved transformation frequencies to greater than 109 transform ant cells per microgram of added DNA, in some instances. E. coli cell lines known in the art to exhibit high transformation efficiency include, for example, DH5α, X1776, and XL1-Blue, among others. Alternatively, microorganisms, including bacteria such as E. coli, may be transformed by electroporation (e.g. Miller and Nickoloff, 1995).

The same procedures may be used to construct and introduce plasmids comprising any of the RBS sequences, e.g. of SEQ ID NO:1-25, into a host cell. Techniques and/or procedures for ligating the described DNA fragments of SEQ ID NO:1 to SEQ ID NO:25 into various other vectors are known and routinely practiced by those of ordinary skill in the art (e.g. Sambrook et al. 1989). Moreover, plasmids suitable for transforming E. coli and/or other bacteria so as to achieve protein expression from cultures of the transformed bacteria are known and regularly used by those of skill in the art.

As used herein, the term “sequence identity”, “sequence similarity” or “homology” is used to describe sequence relationships between two or more nucleotide sequences or amino acid sequences. The percentage of “sequence identity” between two sequences is determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. These terms and descriptions are well defined in the art and are easily understood by those of ordinary skill in the art.

The nucleotide sequences of SEQ ID NOs 1-25 and 30-87 (FIG. 2) each comprise RBS sequences, as well as flanking sequence. Some of these sequences allow expression of bST from the native cDNA when included as an expression element in an expression construct or vector. Bovine somatotropin production was induced under standard growth conditions (Bogosian et al. 1989) and the bST expression level was determined. Vectors comprising SEQ ID NO: 1 through 25 expressed bST from the native cDNA at levels of about 300 milligrams per liter (“mg/l”). Many of these have bST yields above 2000 mg/l, or even 4500 mg/l or higher, using the fermentation conditions described (Bogosian et al., 1989). Under such fermenter vessel culture conditions, the high cell densities achieved and the corresponding levels of expressed proteins are about 100-fold higher than in standard shake flask culture conditions.

It is notable that most of the ribosome binding sites that expressed bST protein at over one gram per liter (FIG. 2) were novel synthetic RBS sequences, and that of 58 natural ribosome binding sites tested (57 native E. coli ribosome binding sites, and the bacteriophage T7 gene 10 ribosome binding site), only 4 expressed bST protein at approximately one gram per liter, or more. A great deal of experimentation was required to identify natural ribosome binding sites effective at the expression of the bST cDNA gene. These natural ribosome binding sites were not known to be effective, nor were they available for standard expression systems, when the early efforts to express the bST cDNA gene were underway. In hindsight, it is thus not surprising that no standard expression systems have been developed until the present invention, that express mature bST protein at grams per liter, or even at any detectable amounts, from a bST cDNA gene.

The RBS sequences that provided the greatest levels of native bST expression have certain features in common. These features are readily apparent when comparing the inoperative prior art RBS sequences of Table 1 (refer to Example 1) with those RBS sequences of the present invention that confer high levels of bST expression (>300 mg/L) (refer to FIG. 2A). One such feature is that the most 3′ sequence (about the last eight nucleotides) is adenine- and thymine-rich; the same sequence portion in the prior art RBS sequences is relatively guanine- and cytosine-rich. Another apparent feature of the inventive RBS sequences is the presence of the subsequence DDAGGDD (D is A, T or G), which is not present in the prior art sequences. The position of this subsequence within the inventive RBS sequences is given by the central G nucleotide, which is about 10 to 13 nucleotides 5′ upstream of the bST start codon. Though the invention is not limited by any sort of mechanism, one would speculate that the capability of the inventive RBS sequences to effect native bST expression is due to these sequence features.

The first two codons of the portion of the bST cDNA gene encoding mature bST protein are GCC followed by TTC, encoding the amino acids alanine followed by phenylalanine. As described in the background section, mature bST protein can begin either with the alanine −1 amino acid followed by the phenylalanine +1 amino acid, or with the phenylalanine +1 amino acid without the preceding alanine −1 amino acid. For the construction of mature bST protein expression systems in recombinant E. coli strains, a methionine initiation codon ATG is positioned at the beginning of the portion of the bST cDNA gene encoding mature bST protein. Depending on the codon immediately following this methionine initiation codon ATG, the bST protein expressed may include a methionine amino acid at position −1. It has been found that if the second codon is an alanine codon, the resulting bST protein contains the amino acid alanine at position −1 followed by phenylalanine at position +1, while if the second codon is a phenylalanine codon, the resulting bST protein contains the amino acid methionine at position −1 followed by phenylalanine at position +1 (Calcott et al., 1988; Warren et al., 1996). In recombinant E. coli strains, the retention of the first methionine amino acid on the bST protein is dependent on the second amino acid encoded by the bST gene being expressed. When the second amino acid is alanine, protein processing in E. coli strains removes the first methionine amino acid from the bST protein, and when the second amino acid is phenylalanine, the methionine is retained (Calcott et al., 1988; Warren et al., 1996). Thus, in recombinant E. coli strains, mature bST protein may be expressed either with the first amino acid being alanine at position −1 followed by phenylalanine at position +1, or with the first amino acid being methionine at position −1 followed by phenylalanine at position +1.

In the examples that follow, the phrase “bST cDNA gene” refers to any of the four naturally occurring variants of the native bST gene, namely SEQ ID NO:27, SEQ ID NO:107, SEQ ID NO:108, or SEQ ID NO:109. The first alanine codon GCC (encoding the alanine amino acid at position −1 of the bST protein) may or may not be present, depending on the bST protein to be expressed.

The phrase “bST protein” refers to any bovine somatotropin protein, including the following polypeptide sequences: SEQ ID NO:29, SEQ ID NO:110, SEQ ID NO:112, or SEQ ID NO:113. As described in these sequences, the first amino acid at position −1 of the bST protein, may be either alanine or methionine, depending on the bST protein being expressed.

### EXAMPLE 1

### Testing of bST Protein Expression Levels from Reported Standard Expression Constructs

For the testing of bST protein expression described below, recombinant E. coli strains were cultured and induced for bST protein expression in fermentation vessels as described in Bogosian et al. (1989). The levels of bST protein in the induced cultures were measured by an HPLC assay with a limit of detection of one milligram per liter, by a Western immunoblot assay with a limit of detection of 100 micrograms per liter, and by a radioimmunoassay with a limit of detection of 100 nanograms per liter. The following systems were tested:

a) An expression system with the novel synthetic hybrid double ribosome binding site (SEQ ID NO:92) and a bST cDNA gene of Schoner et al (e.g. Schoner et al., 1984; European Patent Application 0316115 A2, and U.S. Pat. Nos. 5,063,158 and 5,192,669) was constructed and tested for bST protein expression. This RBS is referred to as the “double” RBS in table 1, and was found to express bST protein at only about 250 milligrams per liter.

b) A set of expression systems comprising the set of seven modified trpE ribosome binding sites (SEQ ID NO:99-105) suggested to express mature bST protein (Miller, European Patent 0047600 B1; Miller, U.S. Pat. No. 6,692,941 B1) was constructed and tested for bST protein expression. All were found not to express bST protein at detectable levels. These seven ribosome binding sites are referred to as “trpE-UCal-1” through “trpE-UCal-7” in Table 1.

c) An expression system with the bacteriophage lambda cro gene ribosome binding site (SEQ ID NO:96) and a bST cDNA gene (Krzyzek et al., 1984; and George et al., 1985), similar to the plasmid designated p27-112-4/C, was constructed and tested for bST protein expression. This ribosome binding site is referred to as “cro-modified” ribosome binding site in Table 1. It was found not to express bST protein at detectable levels.

TABLE 1

| | |
|---|---|
| | bST protein expression |
| SEQ ID NO | (mg per liter) |

| | | | |
|---|---|---|---|
| 92 | | TCTAGAGGGTATTAATAATCTATCGATTAAATAAGGAGGAATAACAT-ATG | 250 |
| | | (Double Ribosome Binding Site) | |
| 93 | trpL | AGTTCACGTAAAAAGGGTATCGACA-ATG | none detected |
| 94 | trpL-Gen | ACGTAAAAAGGGTATCTAGAATTCT-ATG | none detected |
| 95 | trpL-Up | AAGTTCACGTTATTAAAAATTAAAGAGGTATATCGATA-ATG | none detected |
| 96 | cro modified | CCATGTACTAAGGAGGTTCAGATCT-ATG | none detected |
| 97 | cII modified | TTGTTATCTAAGGAAGTACTTACAT-ATG | none detected |
| 98 | ner | TACAAAACTTAGGAGGGTTTTTACC-ATG | none detected |
| 99 | trpE-UCal-1 | AAATTAGAGAATAACCCGGATCCGG-ATG | none detected |
| 100 | trpE-UCal-2 | AAAATTAGAGAATAACCGGATCCGG-ATG | none detected |
| 101 | trpE-UCal-3 | CAAAATTAGAGAATACCGGATCCGG-ATG | none detected |
| 102 | trpE-UCal-4 | ACAAAATTAGAGAATCCGGATCCGG-ATG | none detected |
| 103 | trpE-UCal-5 | AACAAAATTAGAGAACCGGATCCGG-ATG | none detected |
| 104 | trpEU-Cal-6 | GAACAAAATTAGAGACCGGATCCGG-ATG | none detected |
| 105 | trpE-UCal-7 | TGAAGAAAATTAGAGCCGGATCCGG-ATG | none detected |

d) An expression system with the bacteriophage lambda cII gene ribosome binding site (SEQ ID NO:97) and a bST cDNA gene (Oppenheim, U.S. Pat. No. 5,059,529) was also tested. While no bST protein expression tests were done with this vector by these workers, the plasmid (designated plasmid p8300-10A) was obtained from the American Type Culture Collection, Manassas, Va., under accession number ATCC 39785 and was found not to express bST protein at detectable levels. This ribosome binding site is referred to as the “cII-modified” ribosome binding site in Table 1.

e) An expression system was constructed with the bacteriophage Mu ner gene ribosome binding site (SEQ ID NO:98) and a bST cDNA gene (Buell, European Patent Application 0103395 A2, and Buell, U.S. Pat. No. 4,693,973). The plasmid (designated plasmid pBGH-(Met-Ala; also referred to as pBGH006) was obtained from the American Type Culture Collection, Manassas, Va., under accession number ATCC 39173, and was found not to express bST protein at detectable levels. This ribosome binding site is referred to as the “ner” ribosome binding site in Table 1.

f) An expression system with the E. coli chromosomal wild-type trpL gene ribosome binding site (SEQ ID NO:93) and a bST cDNA gene, designated plasmid pTrp-BStm3, or with a modified trpL ribosome binding site (SEQ ID NO:95) and a bST cDNA gene, designated pAT-BSt102, (Tomich, International Patent Application WO 88/06186; Tomich, European Patent 0418219 B1; and Tomich, U.S. Pat. Nos. 5,240,837 5,268,284) has been reported. Similar plasmid expression systems with the wild-type or the modified trpL gene ribosome binding sites and bST cDNA genes were constructed and tested for bST protein expression, and both were found not to express bST protein at detectable levels. These ribosome binding sites are referred to as the “trpL” and “trpL-Up” ribosome binding sites in Table 1.

g) An expression system with a modified trpL gene ribosome binding site (SEQ ID NO:94) and a bST cDNA gene, designated plasmid pBGH33, has been reported (de Boer, European Patent 0075444 B1; de Boer, European Patent 0278121 B1; and de Boer, U.S. Pat. Nos. 4,880,910, 5,254,463, 5,260,201, and 5,489,529). A similar plasmid expression system with the modified trpL gene ribosome binding site and a bST cDNA gene was constructed and tested for bST protein expression. It was found not to express bST protein at detectable levels. This ribosome binding site is referred to as the “trpL-Gen” ribosome binding site in Table 1.

Schoner and coworkers reported a standard expression system designed to express bST protein from a bST cDNA gene, designated as plasmid pCZ108, that reportedly expressed bST protein at about 1.7% of total cell protein (Schoner et al., 1984). However, in a subsequent publication (Hsiung & McKellar, 1987), they reported that pCZ108 did not employ a bST cDNA gene, but rather a modified bST gene with a silent alteration of the codon for leucine +6 from the cDNA codon TTG to the cognate, but non-cDNA, codon CTG. Furthermore, in another paper, they described a plasmid designated pCZ140 with a bST cDNA gene and found no detectable amount of bST protein produced by cultures harboring pCZ140 (Schoner et al., 1986).

Thus, no standard expression systems (that is, employing a promoter and a single ribosome binding site) have been described in the literature that express detectable amounts of bST protein from a bST cDNA gene. Such systems that were reported to have expressed some detectable bST protein from a bST cDNA gene (with reported bST protein expression levels from “poor” to 1% of total cell protein) were re-tested and found not to express bST protein at detectable levels. The apparent discrepancies may be attributed to difficulties in detecting such low quantities of bST protein or in distinguishing bona fide bST protein from false background signals, using the analytical techniques of the 1980's. In any event, bST protein expression levels at only 1% of total cell protein would yield only about 120 milligrams of bST protein per liter under the high cell densities achieved in the fermenter vessels employed for these tests. In standard shake flask cultures, with cell densities about 100-fold less than in the fermenter vessels, bST protein expression levels at only 1% of total cell protein would yield only about one milligram of bST protein per liter.

For comparison purposes, the commercially useful expression plasmids containing a modified bST gene with silent alterations in the beginning of the bST gene, namely pBGH1 (de Boer, U.S. Pat. Nos. 4,880,910, 5,254,463, 5,260,201, and 5,489,529; Calcott et al., 1988; Kane et al., 1991) and pXT709 (Bogosian, U.S. Pat. No. 6,828,124) express bST protein at levels of about 6000 milligrams per liter. Also for comparison, the non-standard expression system comprising a synthetic hybrid double ribosome binding site (Schoner and co-workers) resulted in bST protein expression from a bST cDNA gene of only about 250 milligrams per liter, as noted in (a) above.

### EXAMPLE 2

### Construction of Novel bST Protein Expression Systems

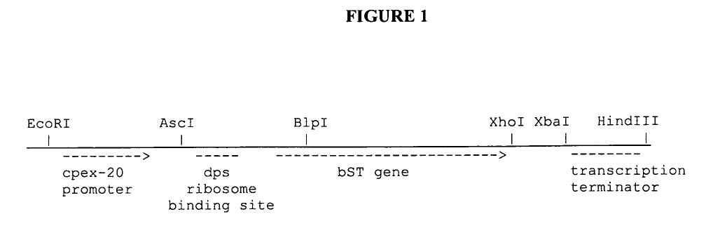

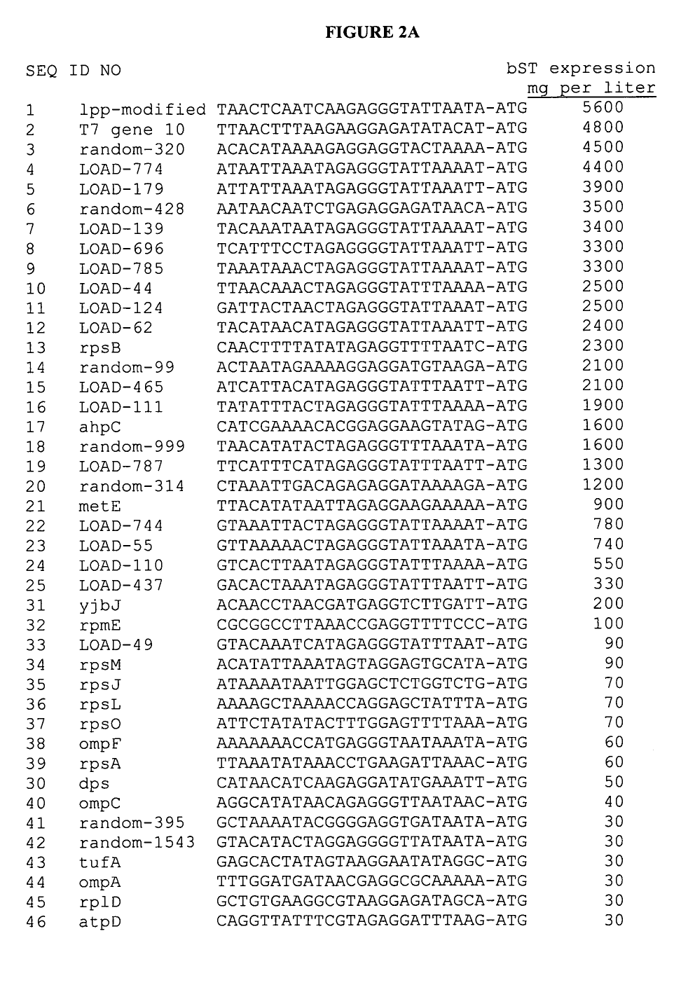

Expression systems for the bST cDNA gene were based on the bST protein expression cassette of plasmid pXT709 (SEQ ID NO:28), a plasmid that has a modified bST gene and that expresses bST protein at levels of about 6000 milligrams per liter (Bogosian, U.S. Pat. No. 6,828,124). The expression system on plasmid pXT709 includes the synthetic cpex-20 promoter (Bogosian, U.S. Pat. No. 6,617,130), the E. coli native dps ribosome binding site, the modified bST gene, and a transcription terminator. There are arrays of restriction sites on the plasmid pXT709 (FIG. 1) that allow the facile replacement of the dps ribosome binding site with any desired ribosome binding site, and the replacement of the modified bST gene with any desired bST gene. There is a unique BlpI site on pXT709 within the bST gene, spanning codons 17-19. Unique EcoRI and AscI sites are positioned upstream of the modified bST gene on pXT709, and unique XhoI and XbaI sites are positioned downstream of the modified bST gene.

First, the modified bST gene on plasmid pXT709 was replaced with an AscI-XbaI fragment containing a dps ribosome binding site and a bST cDNA gene to yield the plasmid pXT737. This bST cDNA gene encodes a Met-Phe-(Leu-126)-bST protein. The sequence of this bST cDNA gene is that given as SEQ ID NO:107 with the first alanine codon being replaced with a methionine initiation codon ATG. Second, other ribosome binding site candidates were identified, and used as synthetic AscI-BlpI DNA fragments (carrying each candidate ribosome binding site and the first 19 codons of the native bST cDNA gene including the BlpI site) to replace the dps ribosome binding site and 5′ end of the bST cDNA gene of pXT737 to yield new plasmids with various ribosome binding sites linked to the native bST cDNA coding sequence.

From compilations of the levels of native proteins in the E. coli cell (VanBogelen et al., 1996; Link et al., 1997), 58 abundant proteins were identified. From the nucleotide sequence of the E. coli genome (Blattner et al., 1997), the ribosome binding sites of these 58 abundant native E. coli proteins were identified. While there may be a variety of reasons a protein may be abundant, it was reasoned that these 58 ribosome binding site sequences would include many very strong ribosome binding sites.

Examination of these 58 native E. coli ribosome binding sites revealed that six of them contained a central 11 nucleotide sequence that shared a high degree of homology to an 11 nucleotide sequence that was termed the LOAD motif. The term LOAD is a mnemonic for those RBS sequences which contained this motif: lpp, ompC (and ompF), atpD (and atpF), and dps. The LOAD sequence motif is TAGAGGGTATT (SEQ ID NO:88), and is positioned from about coordinates −5 through −15 with respect to the ATG start codon of these six genes:

| | | |
|---|---|---|
| LOAD sequence | TAGAGGGTATT | (SEQ ID NO:88) |
| motif: | | |
| lpp | TAACTCAATCTAGAGGGTATTAATA-ATG | (SEQ ID NO:89) |
| ompF | AAAAAAACCATGAGGGTAATAAATA-ATG | (SEQ ID NO:38) |
| ompC | AGGCATATAACAGAGGGTTAATAAC-ATG | (SEQ ID NO:40) |
| atpF | GTTAACTAAATAGAGGCATTGTGCT-ATG | (SEQ ID NO:59) |
| atpD | CAGGTTATTTCGTAGAGGATTTAAG-ATG | (SEQ ID NO:46) |
| dps | CATAACATCAAGAGGATATGAAATT-ATG | (SEQ ID NO:30) |

A randomized synthetic ribosome binding site fragment was designed that contained the LOAD sequence motif flanked by randomized sequences:

| | |
|---|---|
| DDWHAHWWHM-TAGAGGGTATT-WAAW -ATG | (SEQ ID NO:90) |
| DDWHAHWWHM-TAGAGGGTATT-WAAWW-ATG. | (SEQ ID NO:111) |

The randomized synthetic ribosome binding site fragment was designed with the spacer between the LOAD sequence motif and the ATG initiation codon being either WAAW or WAAWW.

Examination of all 58 of the native E. coli ribosome binding sites for the abundant native E. coli proteins revealed a consensus sequence that was used in the design of another randomized synthetic ribosome binding site fragment:

| | |
|---|---|
| NNWMANWNWMNNRRRGGWNNWWANA-ATG | (SEQ ID NO:91) |

These randomized synthetic ribosome binding site sequences use the standard IUPAC one letter codes for the standard and mixed bases, where D stands for A or T or G, W stands for A or T, H stands for A or T or C, M stands for A or C, R stands for A or G, Y stands for C or T, and N stands for A or G or C or T. The last W in the random LOAD ribosome binding site sequence, immediately before the ATG initiation codon, is a position that was randomized so as to have W present in half of the randomized fragments, and omitted in half of the randomized fragments.

From these randomized synthetic ribosome binding site sequences, additional functional ribosome binding sites were identified and studied further. These were termed either LOAD ribosome binding sites, or random ribosome binding sites. There were 16 novel synthetic LOAD ribosome binding sites identified, and 8 novel random ribosome binding sites identified.

In addition, the bacteriophage T7 gene 10 ribosome binding site (SEQ ID NO:2) was also tested, as it is a particularly strong ribosome binding site (Olins, U.S. Pat. No. 5,232,840; Olins et al., 1988; and Olins and Rangwala, 1989).

Thus, a total of 83 ribosome binding sites were tested, namely 57 native E. coli ribosome binding sites, a modified E. coli lpp ribosome binding site, 16 novel synthetic LOAD ribosome binding sites, 8 novel synthetic random ribosome binding sites, and the bacteriophage T7 gene 10 ribosome binding site. The E. coli native lpp ribosome binding site was not tested, but rather a modified version in which the first T nucleotide in the central LOAD motif sequence TAGAGGGTATT (SEQ ID NO:88) was changed to an A nucleotide (as seen in SEQ ID NO:1). This modified lpp ribosome binding site was tested as other work had indicated it to be a stronger ribosome binding site than the native lpp ribosome binding site.

### EXAMPLE 3

### Levels of bST Protein Obtained from Novel Expression Systems

The 83 new plasmids, with the 83 different candidate ribosome binding sites and the bST cDNA gene, were transformed into the E. coli K-12 host strain LBB427. Strain LBB427 is nearly identical to the standard wild-type E. coli K-12 strain W3110; the only difference being that strain LBB427 has a mutation in the fhuA gene. This mutation does not affect bST protein expression in any way. The strains were cultured in fermentation vessels as described in Bogosian et al. (1989), except that 50 ppm of nalidixic acid was used as the inducer of the cpex-20 promoter (Bogosian, U.S. Pat. Nos. 6,828,124 and 6,617,130). The levels of bST protein in the induced cultures were measured by an HPLC assay with a limit of detection of one milligram per liter.

The results, listed in FIG. 2, show that 25 of the 83 tested ribosome binding sites expressed bST protein at levels over 250 milligrams per liter, that is, higher than any standard expression system known to date. These 25 ribosome binding sites included 15 LOAD ribosome binding sites, 5 random ribosome binding sites, the E. coli native rpsB, ahpC, and metE ribosome binding sites, the bacteriophage T7 gene 10 ribosome binding site, and the modified lpp ribosome binding site.

Nine of the tested ribosome binding sites expressed bST protein at levels over 3000 milligrams per liter, and ranging as high as 5600 milligrams per liter. None of the bST cDNA expression systems produced as much bST protein as the commercially useful expression plasmids containing a modified bST gene with silent alterations in the beginning of the bST gene, namely pBGH1 (de Boer, U.S. Pat. Nos. 4,880,910, 5,254,463, 5,260,201, and 5,489,529; Calcott et al., 1988; Kane et al., 1991) and pXT709 (Bogosian, U.S. Pat. No. 6,828,124), plasmids that both express bST protein at levels of about 6000 milligrams per liter. However, the present invention provides for the first time standard expression systems employing a bST cDNA gene that yield bST protein at several grams per liter.

A great deal of effort was expended to test 83 ribosome binding sites. It is notable that most of the ribosome binding sites that expressed bST protein at over one gram per liter were novel synthetic RBS sequences, and that of 58 natural ribosome binding sites tested (the 57 native E. coli ribosome binding sites, and the bacteriophage T7 gene 10 ribosome binding site), only 3 expressed bST protein at over one gram per liter. A great deal of experimentation was also required to identify natural ribosome binding sites effective at the expression of the bST cDNA gene. These natural ribosome binding sites were not known to be effective, nor were they available for standard expression systems, when the early efforts to express the bST cDNA gene were underway. In hindsight, it is thus not surprising that no standard expression systems have been developed until the present invention, that express bST protein at grams per liter, or even at any detectable amounts, from a bST cDNA gene.

### EXAMPLE 4

### Consensus Ribosome Binding Site

Examination and alignment of RBS sequences effective in expressing bST protein from the bST cDNA gene at a level of at least 300 milligrams per liter resulted in identification of the following consensus RBS sequence:

| | |
|---|---|
| MHWHHWHDWHMDRGAGGRWRTWWRM | (SEQ ID NO:106) |

wherein the consensus sequence is followed by a spacer of 0-2 nucleotides, the first being W and the second being T, followed by the ATG start codon.

RBS sequences that vary from that shown in SEQ ID NO:106 by 1, 2, 3, or 4 nucleotides and that are still effective for expressing bST protein were also identified (FIG. 2). One of skill in the art could make other RBS sequences within the scope of the claims by substituting nucleotides of the consensus sequence and screening them for activity according to the procedures in the working examples.

While the materials and methods of this invention have been described in terms of preferred embodiments, it will be apparent to those of skill in the art that variations may be applied to the method described herein without departing from the concept, spirit and scope of the invention. All such similar substitutes and modifications apparent to those skilled in the art are deemed to be within the spirit, scope and concept of the invention

### REFERENCES CITED

The following references are herein incorporated by reference in there entirety:
Aviv, H., A. Rosner, E. Keshet, and M. Gorecki. Microorganism adapted to the production of bovine somatotropic hormone and a modified hormone product. French patent application 19815731.
Aviv, H., A. Rosner, E. Keshet, and M. Gorecki. Production of bovine growth hormone by microorganisms. UK Patent GB 2073245 B.
Aviv, H., E. Keshet, M. Gorecki, and A. Rosner. Production of bovine growth hormone by microorganisms. U.S. Pat. No. 6,229,003 B1.
Baxter, J. D., H. M. Goodman, J. A. Martial, and R. A. Hallewell. A DNA transfer vector for human pre-growth hormone, a microorganism transformed thereby, and a method of cloning therefor. European Patent 0020147 B2.
Blattner, F. R., G. Plunkett III, C. A. Bloch, N. T. Perna, V. Burland, M. Riley, J. Collado-Vides, J. D. Glasner, C. K. Rode, G. F. Mayhew, J. Gregor, N. W. Davis, H. A. Kilpatrick, M. A. Goeden, D. J. Rose, B. Mau, and Y. Shao. 1997. The complete genome sequence of Escherichia coli K-12. Science 277: 1453-1462.
Bogosian, G., B. N. Violand, E. J. Dorward-King, W. E. Workman, P. E. Jung, and J. F. Kane. 1989. Biosynthesis and incorporation into protein of norleucine by Escherichia coli. J. Biol. Chem. 264: 531-539.
Bogosian, G., J. P. O'Neil, and K. C. Terlesky. DNA construct for regulating the expression of a polypeptide coding sequence in a transformed bacterial host cell. U.S. Pat. No. 6,617,130.
Bogosian, G., J. P. O'Neil, and N. D. Aardema. Recombinant DNA vectors for expression of somatotropins. U.S. Pat. No. 6,828,124.
Buell, G. N. 1982. DNA sequences, recombinant DNA molecules, and processes for producing bovine growth hormone-like polypeptides in high yield. European Patent Application 0103395 A2.
Buell, G. N. DNA sequences, recombinant DNA molecules, and processes for producing bovine growth hormone-like polypeptides in high yield. U.S. Pat. No. 4,693,973.
Calcott, P. H., J. F. Kane, G. G. Krivi, and G. Bogosian. 1988. Parameters affecting production of bovine somatotropin in Escherichia coli fermentations. Dev. Indust. Micro. 29: 257-266.
Chasov, V. V., A. A. Deev, I. S. Masulis, and O. N. Ozoline 2002 Distribution and functional significance of A/T tracts in promoter sequences of Escherichia coli. Mol. Biol. 36: 537-542.
Cho, J. M., T. H. Lee, H. H. Chung, Y. B. Lee, T. G. Lee, Y. W. Park and K. B. Han. Method for the production of bovine growth hormone using a synthetic gene. U.S. Pat. No. 5,366,876.
Choi, J. W., and S. Y. Lee. 1996. Optimization of the bovine growth hormone gene expression in E. coli. Mol. Cells 6: 712-718.
Choi, J. W., K. S. Ra, and Y. S. Lee. 1999. Enhancement of bovine growth hormone gene expression by increasing the plasmid copy number. Biotechnol. Lett. 21: 1-5.
Dayhoff, M. O. 1976. Atlas of protein sequence and structure, Volume 5, Supplement 2. Somatropin-bovine. Page 139.
de Boer, H. A., H. L. Heyneker, and P. H. Seeburg. DNA for expression of bovine growth hormone. U.S. Pat. No. 5,489,529.
de Boer, H. A., H. L. Heyneker, and P. H. Seeburg. Method for expression of bovine growth hormone. U.S. Pat. No. 5,254,463.
de Boer, H. A., H. L. Heyneker, and P. H. Seeburg. Methods and products for facile microbial expression of DNA sequences. U.S. Pat. No. 5,260,201.
de Boer, H. A., H. L. Heyneker, and P. H. Seeburg. Terminal methionyl bovine growth hormone and its use. U.S. Pat. No. 4,880,910.
de Boer, H. A., P. H. Seeburg, and H. L. Heyneker. Methods and products for facile microbial expression of DNA sequences. European Patent 0075444 B1.
de Boer, H. A., P. H. Seeburg, and H. L. Heyneker. Microbially produced bovine growth hormone and its use. European Patent 0278121 B1.
Efstratiadis, A., and L. VIIIa-Komaroff. 1979. Cloning of double-stranded cDNA. Pages 15-36, in Setlow, J. K., and A. Hollaender (eds.) “Genetic engineering, principles and methods” Volume 1. Plenum Press, NY.
Fellows, Jr., R. E., A. D. Rogol, and A. Mudge. 1971. Structural studies on bovine growth hormone, page 3 in “Second international symposium on growth hormone”, Excerpta Medica, Amsterdam.
Fellows, Jr., R. E., A. D. Rogol, and A. Mudge. 1972. Structural studies on bovine growth hormone. pp. 42-54. In Pecile, A., and E. E. Muller (eds) “Growth and growth hormone. Proceedings of the second international symposium on growth hormone, Milan, May 5-7, 1971.” Excerpta Medica, Amsterdam.
Fellows, Jr., R. E., and A. D. Rogol. 1969. Structural studies on bovine growth hormone. I. Isolation and characterization of cyanogen bromide fragments. J. Biol. Chem. 244: 1567-1575.
Fiddes, J. C., and H. M. Goodman. 1979. Isolation, cloning and sequence analysis of the cDNA for the alpha-subunit of human chorionic gonadotropin. Nature 281: 351-356.
Franke, A. E. 1983. Expression plasmids for improved production of heterologous protein in bacteria. European Patent 0147178 B1.
Franke, A. E. Expression plasmids for improved production of heterologous protein in bacteria. U.S. Pat. No. 4,935,370.
Franke, A. E. Expression plasmids for improved production of heterologous protein in bacteria. U.S. Pat. No. 5,955,297.
French Patent Application 7815596, “Recombinant DNA transfer vector and microorganism containing a gene from a higher organism”.
George, H. J., J. J. L'Italien, W. P. Pilacinski, D. L. Glassman, and R. A. Krzyzek. 1985. High-level expression in Escherichia coli of biologically active bovine growth hormone. DNA 4: 273-281.
George, H. J., R. A. Krzyzek, L. W. Enquist, and R. J. Watson. Co-aggregate purification of proteins. U.S. Pat. No. 4,673,641.
Glassman, D. L., W. P. Pilacinski, H. J. George, R. A. Krzyzek, J. S. Salstrom, and D. Newman. The cloning and expression of nucleotide sequences encoding bovine growth hormone. European Patent Application 0111814 A2.
Goeddel, D. V., and H. L. Heyneker. Method of constructing a replicable cloning vehicle having quasi-synthetic genes. U.S. Pat. No. 4,342,832.
Goodman, H. M., and R. J. MacDonald. 1979. Cloning of hormone genes from a mixture of cDNA molecules. Methods in Enzymology 68: 75-90.
Goodman, H. M., J. Shine, and P. H. Seeburg. Recombinant DNA transfer vectors. U.S. Pat. No. 4,363,877.
Gordon, D. F., D. P. Quick, C. R. Erwin, J. E. Donelson, and R. A. Maurer. 1983. Nucleotide sequence of the bovine growth hormone chromosomal gene. Mol. Cellular Endocrinol. 33: 81-95.
Graf, L., and C. H. Li. 1974. On the primary sequence of pituitary bovine growth hormone. Biochem. Biophys. Res. Commun. 56: 168-176.
Gubbins, E. J., R. A. Maurer, J. L. Hartley, and J. E. Donelson. 1979. Construction and analysis of recombinant DNAs containing a structural gene for rat prolactin. Nucleic Acids Res. 6: 915-930.
Hanahan et al., 1983. Studies on transformation of Escherichia coli with plasmids. J. Mol. Biol. 166: 557-580.
Hanahan, D.; et al., 1991. Plasmid transformation of Escherichia coli and other bacteria. Meth. Enzymol., 20:63-113.
Harpold, M. M., P. R. Dobner, R. M. Evans, and F. C. Bancroft. 1978. Construction and identification by positive hybridization-translation of a bacterial plasmid containing a rat growth hormone structural gene sequence. Nucleic Acids Res. 5: 2039-2053.
Hershberger, C. L., and J. L. Larson. Plasmid pHKY334, an expression vector for EK-bGH and host cells transformed therewith. U.S. Pat. No. 5,395,761.
Hsiung, H. M., and W. C. MacKellar. 1987. Expression of bovine growth hormone derivatives in Escherichia coli and the use of the derivatives to produce natural sequence growth hormone by cathepsin C cleavage. Methods in Enzymology 153: 390-401.
Israeli Patent Application 59690, “Production of bovine growth hormone by microorganisms”.
Itakura, K. Recombinant DNA cloning vehicle. U.S. Pat. No. 4,356,270
Itakura, K., and A. D. Riggs. Method for microbial polypeptide expression. UK Patent Application 2007676.
Itakura, K., and A. D. Riggs. Methods and means for microbial polypeptide expression. U.S. Pat. No. 5,221,619.
Itakura, K., and A. D. Riggs. Recombinant cloning vehicle microbial polypeptide expression. U.S. Pat. No. 4,704,362.
Kane, J. F., and G. Bogosian. 1987. Bovine somatotropin production: Selecting the best host-vector system. BioPharm Manufacturing 1: 26-51.
Kane, J. F., S. M. Balaban, and G. Bogosian. 1991. Commercial production of bovine somatotropin in Escherichia coli. In Sikes, C. S., and A. P. Wheeler (ed.) “Surface reactive peptides and polymers. Discovery and commercialization” pp 186-200. Amer. Chem. Soc., Washington, D.C.
Keshet, E., A. Rosner, Y. Bernstein, M. Gorecki, and H. Aviv. 1981. Cloning of bovine growth hormone gene and its expression in bacteria. Nucleic Acids Research 9: 19-30.
Krzyzek, R. A., H. J. George, T. Kempe, J. J. L'Italien, D. K. Anderson, D. F. Harbrecht, D. E. Smolin, and G. A. Ellestad. 1984. High level expression of bovine growth hormone in E. coli: Modification of gene sequences to enhance translation. Abstracts of the Annual Meeting of the American Society for Microbiology. Abstract H 105, page 109.
Li, C. H., and L. Ash. 1953. The nitrogen terminal end-groups of hypophyseal growth hormone. J. Biol. Chem. 203: 419-424.
Lingappa, V. R., A. Devillers-Thiery, and G. Blobel. 1977. Nascent prehormones are intermediates in the biosynthesis of authentic bovine pituitary growth hormone and prolactin. Proc. Natl. Acad. Sci. 74: 2432-2436.
Link, A. J., K. Robison, and G. M. Church. 1997. Comparing the predicted and observed properties of proteins encoded in the genome of Escherichia coli K-12. Electrophoresis 18: 1259-1313.
Lisser, S., and H. Margalit. 1993. Compilation of E. coli mRNA promoter sequences. Nucleic Acids Res. 21:1507-1516.
Lucy, M. C., S. D. Hauser, P. J. Eppard, G. G. Krivi, J. H. Clark, D. E. Baumann, and R. J. Collier. 1993. Variants of somatotropin in cattle: Gene frequencies in major dairy breeds and associated milk production. Domestic Animal Endocrinology 10: 325-333.
Martial, J. A., R. A. Hallewell, J. D. Baxter, and H. M. Goodman. 1979. Human growth hormone: Complementary DNA cloning and expression in bacteria. Science 205: 602-607.
Mayne, N. G., H. M. Hsiung, J. D. Baxter, and R. M. Belagaje. 1984. Direct expression of human growth hormone in Escherichia coli with the lipoprotein promoter, pp. 135-143. In Bollon, A. P. (ed.) “Recombinant DNA products: Insulin, interferon, and growth hormone.” CRC Press, Boca Raton, Fla.
Mayne, N. G., J. P. Burnett, R. Belegaje, and H. M. Hsiung. Cloning vectors for expression of exogenous proteins. European Patent 0095361 B1.
Miller E. M., and J. A. Nickoloff. 1995. Escherichia coli electrotransformation. Meth. Mol. Biol. 47:105-113.
Miller, W. L. 1979. Use of recombinant DNA technology for the production of polypeptides. Pages 153-174, in Petricciani, J. C., H. E. Hopps, and P. J. Chapple (eds.) “Cell substrates: Their use in the production of vaccines and other biologicals. Plenum Press, NY.
Miller, W. L., J. A. Martial, and J. D. Baxter. 1980a. Molecular cloning of gene sequences coding for bovine growth hormone and prolactin. Pediatrics Research, volume 14, number 4, part 2 of 2 parts, abstract 335.
Miller, W. L., J. A. Martial, and J. D. Baxter. 1980b. Molecular cloning of DNA complementary to bovine growth hormone mRNA. J. Biol. Chem. 255: 7521-7524.
Miller, W. L., J. A. Martial, and J. D. Baxter. Bovine growth hormone. U.S. Pat. No. 6,692,941 B1.
Miller, W. L., J. A. Martial, and J. D. Baxter. Bovine pre-growth and growth hormone. European Patent 0047600 B1.
Olins, P. O. Enhanced protein production in bacteria by employing a novel ribosome binding site. U.S. Pat. No. 5,232,840.
Olins, P. O., and S. H. Rangwala. 1989. A novel sequence element derived from bacteriophage T7 mRNA acts as an enhancer of translation of the lacZ gene in Escherichia coli. J. Biol. Chem. 264: 16973-16976.
Olins, P. O., C. S. Devine, S. H. Rangwala, and K. S. Kavka. 1988. The T7 phage gene 10 leader RNA, a ribosome-binding site that dramatically enhances the expression of foreign genes in Escherichia coli. Gene 73: 227-235.
Olson, E. R., and E. B. Watson. Expression vectors. International Patent Application WO 89/07141.
Olson, E. R., M. K. Olsen, P. S. Kaytes, H. P. Patel, S. K. Rockenbach, E. B. Watson, and C.-S. C. Tomich. 1987. Translation initiation controls bovine growth hormone gene expression in E. coli. Abstracts of the Annual Meeting of the American Society for Microbiology, page 155, abstract H-97.
Oppenheim, A. B., and G. Locker. 1984. Stabilized expression vectors containing lambda PL promoter and the gene for the cI434 repressor, plasmids containing the vectors, hosts containing the plasmids and related methods. U.S. Pat. No. 5,059,529.
Pena, C., A. C. Paladini, J. M. Dellacha, and J. A. Santome. 1969. Evidence for nonallelic origin of the two chains in ox growth hormone. Biochim. Biophys. Acta 194: 320-321.
Pena, C., A. C. Paladini, J. M. Dellacha, and J. A. Santome. 1970. Structural studies on ovine growth hormone. Cyanogen bromide fragments: N- and C-terminal sequences. Eur. J. Biochem. 17: 27-31.
Reid, E. 1951. Relative importance of free α- and ε-amino groups for the biological activity of the growth hormone. Nature 168: 955.
Roberts, J. L., P. H. Seeburg, J. Shine, E. Herbert, J. D. Baxter, and H. M. Goodman. 1979. Corticotropin and beta-endorphin: Construction and analysis of recombinant DNA complementary to mRNA for the common precursor. Proc. Natl. Acad. Sci. 76: 2153-2157.
Roskam, W. G. and F. Rougeon. 1979. Molecular cloning and nucleotide sequence of the human growth hormone structural gene. Nucleic Acids Research 7: 305-320.
Rottman, F. M., and J. H. Nilson. Process for cloning bovine growth hormone gene, and plasmids and plasmid hosts for use therein. European Patent Application 0067026 A1.
Rubtsov, P. M., B. K. Chemov, V. G. Gorbulev, A. S. Parsadanyan, P. S. Sverdlova, V. V. Chupeeva, Y. B. Golova, N. V. Batchikova, G. S. Zhvirblis, K. G. Skryabin, and A. A. Baev. 1985. Genetic engineering of peptide hormones. Mol. Biol. 19: 226-235.
Rutter, W. J., H. M. Goodman, A. Ullrich, J. Shine, J. M. Chirgwin, R. L. Pictet, and P. H. Seeburg. Recombinant DNA transfer vector and microorganism containing a gene from a higher organism. South African Patent Application 782805.
Rutter, W. J., H. M. Goodman, A. Ullrich, J. Shine, J. M. Chirgwin, R. L. Pictet, and P. H. Seeburg. Recombinant DNA transfer vector and microorganism containing a gene from a higher organism. British Patent 1565190.
Rutter, W. J., H. M. Goodman, and J. D. Baxter. A DNA transfer vector, a microorganism modified by said vector and the synthesis of a eukaryotic protein by the modified microorganism. European Patent 0012494 B1.
Sambrook et al., (ed.) 1989. Molecular Cloning, Cold Spring Harbor Laboratory Press, Cold Spring Harbor, N.Y.
Santome, J. A., J. M. Dellacha, A. C. Paladini, C. Pena, M. J. Biscoglio, S. T. Daurat, E. Poskus, and C. E. M. Wolfenstein. 1973. Primary structure of bovine growth hormone. Eur. J. Biochem. 37: 164-170.
Santome, J. A., J. M. Dellacha, A. C. Paladini, C. E. M. Wolfenstein, C. Pena, E. Poskus, S. T. Daurat, M. J. Biscoglio, Z. M. M. De Sese, and A. V. F. De Sanguesa. 1971. The amino acid sequence of bovine growth hormone. FEBS Lett. 16: 198-200.
Schoner, B. E., and R. G. Schoner. Method to produce recombinant proteins using an expression vector comprising transcriptional and translational activating sequences. U.S. Pat. No. 5,192,669.
Schoner, B. E., and R. G. Schoner. Novel vectors and expression sequences for production of polypeptides. European Patent Application 0316115 A2.
Schoner, B. E., and R. G. Schoner. Recombinant DNA expression vector comprising both transcriptional and translational activating sequences. U.S. Pat. No. 5,063,158.
Schoner, B. E., H. M. Hsiung, R. M. Belagaje, N. G. Mayne, and R. G. Schoner. 1984. Role of mRNA translational efficiency in bovine growth hormone expression in Escherichia coli. Proc. Natl. Acad. Sci. 81: 5403-5407.
Schoner, B. E., R. M. Belagje, and R. G. Schoner. 1986. Translation of a synthetic two-cistron mRNA in Escherichia coli. Proc. Natl. Acad. Sci. 83: 8506-8510.
Schoner, B. E., R. M. Belagje, and R. G. Schoner. 1987. Expression of eukaryotic genes in Escherichia coli with a synthetic two-cistron system. Methods in Enzymology 153: 401-416.
Schoner, R. G., and B. E. Schoner. Recombinant DNA expression vectors and method for gene expression. European Patent Application 0154539 A2.
Seavey, B. K., R. N. P. Singh, U. J. Lewis, and I. I. Geschwind. 1971. Bovine growth hormone: Evidence for two allelic forms. Biochem. Biophys. Res. Commun. 43: 189-195.
Seeburg, P. H., J. Shine, J. A. Martial, A. Ullrich, J. D. Baxter, and H. M. Goodman. 1977b. Nucleotide sequence of part of the gene for human chorionic somatomammotropin: Purification of DNA complementary to predominant mRNA species. Cell 12: 157-165.
Seeburg, P. H., J. Shine, J. A. Martial, J. D. Baxter, and H. M. Goodman. 1977a. Nucleotide sequence and amplification in bacteria of structural gene for rat growth hormone. Nature 270: 486-494.
Seeburg, P. H., S. Sias, J. Adelman, H. A. de Boer, J. Hayflick, P. Jhurani, D. V. Goeddel, and H. L. Heyneker. 1983. Efficient bacterial expression of bovine and porcine growth hormones. DNA 2: 37-45.
Sekine, S., T. Mizukami, T. Nishi, Y. Kuwana, A. Saito, M. Sato, S. Itoh, and H. Kawauchi. 1985. Cloning and expression of cDNA for salmon growth hormone in Escherichia coli. Proc. Natl. Acad. Sci. USA 82: 4306-4310.
Shine, J., P. H. Seeburg, J. A. Martial, J. D. Baxter, and H. M. Goodman. 1977. Construction and analysis of recombinant DNA for human chorionic somatomammotropin. Nature 270: 494-499.
Souza, L. M., T. C. Boone, D. Murdock, K. Langley, J. Wypych, D. Fenton, S. Johnson, P. H. Lai, R. Everett, R.-Y. Hsu, and R. Bosselman. 1984. Application of recombinant DNA technologies to studies on chicken growth hormone. J. Experimental Zoology 232: 465-473.
Szoka, P., A. B. Schreiber, H. Chan, and J. Murthy. 1986. A general method for retrieving the components of a genetically engineered fusion protein. DNA 5: 11-20.
Taylor, J. M. 1979. The isolation of eukaryotic messenger RNA. Ann. Rev. Biochem. 48: 681-717.
Tomich, C.-S. C., and M. K. Olsen. Ribosome binding site. U.S. Pat. No. 5,268,284.
Tomich, C.-S. C., E. R. Olson, and J. E. Mott. cDNAs encoding somatotropin, expression vectors and hosts. Interntional Patent Applicaton WO 88/06186.
Tomich, C.-S. C., E. R. Olson, and J. E. Mott. cDNAs encoding somatotropin, expression vectors and hosts. European Patent 0418219 B1.
Tomich, C.-S. C., E. R. Olson, and J. E. Mott. cDNAs encoding somatotropin, expression vectors, and hosts. U.S. Pat. No. 5,240,837.
Tomich, C.-S. C., E. R. Olson, M. K. Olsen, P. S. Kaytes, S. K. Rockenbach, and N. T. Hatzenbuhler. 1989. Effect of nucleotide sequences directly downstream from the AUG on the expression of bovine somatotropin in E. coli. Nucleic Acids Res. 17: 3179-3197.
VanBogelen, R. A., K. Z. Abshire, A. Pertsemlidis, R. L. Clark, and F. C. Neidhardt. 1996. Gene-protein database of Escherichia coli K-12, edition 6. pp. 2067-2117. In Neidhardt, F. C., R. Curtiss, J. L. Ingraham, E. C. C. Lin, K. B. Low, B. Magasanik, W. S. Reznikoff, M. Riley, M. Schaechter, and H. E. Umbarger (eds) “Escherichia coli and Salmonella cellular and molecular biology.” ASM Press, Washington, D.C.
Wallis, M. 1969. The N-terminus of ox growth hormone. FEBS Lett. 3: 118-120.
Wallis, M. 1973. The primary sequence of bovine growth hormone. FEBS Lett. 35: 11-14.
Wallis, M., and R. V. Davies. 1976. Studies on the chemistry of bovine and rat growth hormones. pp. 1-13. In Pecile, A., and E. E. Muller (eds) “Growth hormone and related proteins. Proceedings of the third international symposium, Milan, Sep. 17-20, 1975.” Excerpta Medica, Amsterdam.
Warren, W. C., K. A. Bentle, M. R. Schlittler, A. C. Schwane, J. P. O'Neil, and G. Bogosian. 1996. Increased production of peptide deformylase eliminates retention of formylmethionine in bovine somatotropin overproduced in Escherichia coli. Gene 174: 235-238.
Watson, E. B., and E. R. Olson. 1987. Plasmid mutations that increase bovine growth hormone gene expression in Escherichia coli. Abstracts of the Annual Meeting of the American Society for Microbiology, page 155, abstract H-99.
Watson, N., and E. R. Olson. 1990. Point mutations in a pBR322-based expression plasmid resulting in increased synthesis of bovine growth hormone in Escherichia coli. Gene 86: 137-144.
Wood, D. C., W. J. Salsgiver, T. R. Kasser, G. W. Lange, E. Rowold, B. N. Violand, A. Johnson, R. M. Leimgruber, G. R. Parr, N. R. Siegel, N. M. Kimack, C. E. Smith, J. F. Zobel, S. M. Ganguli, J. R. Garbow, G. Bild, and G. G. Krivi. 1989. Purification and characterization of pituitary bovine somatotropin. J. Biol. Chem. 264: 14741-14747.
Woychik, R. P., S. A. Camper, R. H. Lyons, S. Horowitz, E. C. Goodwin, and F. M. Rottman. 1982. Cloning and nucleotide sequencing of the bovine growth hormone gene. Nucleic Acids Research 10: 7197-7210.
Wu, R. (ed.) (1979) “Recombinant DNA” Methods in Enzymology, volume 68. Academic Press, NY.

What is claimed is:

1. A nucleic acid sequence comprising SEQ ID NO:106 operably linked to a native bovine somatotropin (bST) cDNA sequence.

2. The nucleic acid sequence of claim 1, further defined as operably linked to a promoter.

3. A nucleic acid sequence comprising SEQ ID NO:8 operably linked to a native bST cDNA sequence.

4. The nucleic acid sequence of claim 3, further defined as operably linked to a promoter.

5. A recombinant construct comprising a ribosome binding site sequence operably linked to a native bST cDNA sequence, wherein
(i) the ribosome binding site sequence contains the subsequence DDAGGDD,
(ii) the central G of the subsequence is located 10 to 13 nucleotides 5′ of the ATG start codon of the cDNA sequence, and
(iii) the 6 to 9 nucleotides between the subsequence and the ATG start codon comprise at least four nucleotides that are adenine or thymine.

6. The recombinant construct of claim 5, further defined as operably linked to a promoter.

7. The recombinant construct of claim 6, wherein the promoter comprises the nucleic acid sequence of SEQ ID NO:26.

8. The recombinant construct of claim 5, wherein the bST cDNA sequence encodes a polypeptide sequence selected from the group consisting of SEQ ID NO:29, SEQ ID NO:110, SEQ ID NO:112 and SEQ ID NO:113.

9. A recombinant construct comprising a ribosome binding site sequence at least 80% identical to SEQ ID NO:106 and operably linked to a native bST cDNA sequence, wherein the last eight nucleotides at the 3′-end of the ribosome binding site sequence comprise at least seven nucleotides that are adenine or thymine.

10. A recombinant construct comprising a ribosome binding site sequence at least 80% identical to SEQ ID NO:106 and operably linked to a native bST cDNA sequence.

11. The recombinant construct of claim 10, further defined as operably linked to a promoter.

12. The recombinant construct of claim 11, wherein the promoter comprises the nucleic acid sequence of SEQ ID NO:26.

13. The recombinant construct of claim 10, wherein the ribosome binding site sequence is at least 84% identical to SEQ ID NO:106.

14. The recombinant construct of claim 10, wherein the ribosome binding site sequence is at least 88% identical to SEQ ID NO:106.

15. The recombinant construct of claim 10, wherein the ribosome binding site sequence is at least 92% identical to SEQ ID NO:106.

16. The recombinant construct of claim 10, wherein the ribosome binding site sequence is at least 96% identical to SEQ ID NO:106.

17. The recombinant construct of claim 10, wherein the ribosome binding site sequence is selected from the group consisting of SEQ ID NOs:1-7 and SEQ ID NOs:9-25.

18. The recombinant construct of claim 10, wherein the ribosome binding site sequence comprises SEQ ID NO:106.

19. The recombinant construct of claim 10, wherein the bST cDNA sequence encodes a polypeptide sequence selected from the group consisting of SEQ ID NO:29, SEQ ID NO:110, SEQ ID NO:112 and SEQ ID NO:113.

20. A transformed host cell comprising the recombinant construct of claim 11.

21. The host cell of claim 20, wherein the host cell is a prokaryotic cell.

22. The host cell of claim 21, wherein the host cell is an E. coli cell.

23. A transformed host cell comprising the nucleic acid sequence of claim 4.

24. The host cell of claim 23, wherein the host cell is a prokaryotic cell.

25. The host cell of claim 24, wherein the host cell is an E. coli cell.

26. A transformed host cell comprising the recombinant construct of claim 6.

27. The host cell of claim 26, wherein the host cell is a prokaryotic cell.

28. The host cell of claim 27, wherein the host cell is an E. coli cell.

29. A method for producing bST in a transformed host cell, comprising:
(a) obtaining the host cell according to claim 20, 23 or 26; and
(b) culturing the host cell under conditions that induce gene expression from the cDNA sequence.


SEQUENCE LISTING (113 sequences)

SEQ ID NO: 1 - DNA, 25 bp, Artificial sequence
  Ribosome binding site
taactcaatcaagagggtattaata

SEQ ID NO: 2 - DNA, 25 bp, Bacteriophage T7
ttaactttaagaaggagatatacat

SEQ ID NO: 3 - DNA, 25 bp, Artificial sequence
  Ribosome binding site
acacataaaagaggaggtactaaaa

SEQ ID NO: 4 - DNA, 25 bp, Artificial sequence
  Ribosome binding site
ataattaaatagagggtattaaaat

SEQ ID NO: 5 - DNA, 25 bp, Artificial sequence
  Ribosome binding site
attattaaatagagggtattaaatt

SEQ ID NO: 6 - DNA, 25 bp, Artificial sequence
  Ribosome binding site
aataacaatctgagaggagataaca

SEQ ID NO: 7 - DNA, 25 bp, Artificial sequence
  Ribosome binding site
tacaaataatagagggtattaaaat

SEQ ID NO: 8 - DNA, 25 bp, Artificial sequence
  Ribosome binding site
tcatttcctagaggggtattaaatt

SEQ ID NO: 9 - DNA, 25 bp, Artificial sequence
  Ribosome binding site
taaataaactagagggtattaaaat

SEQ ID NO: 10 - DNA, 25 bp, Artificial sequence
  Ribosome binding site
ttaacaaactagagggtatttaaaa

SEQ ID NO: 11 - DNA, 25 bp, Artificial sequence
  Ribosome binding site
gattactaactagagggtattaaat

SEQ ID NO: 12 - DNA, 25 bp, Artificial sequence
  Ribosome binding site
tacataacatagagggtattaaatt

SEQ ID NO: 13 - DNA, 25 bp, Escherichia coli
caacttttatatagaggttttaatc

SEQ ID NO: 14 - DNA, 25 bp, Artificial sequence
  Ribosome binding site
actaatagaaaaggaggatgtaaga

SEQ ID NO: 15 - DNA, 25 bp, Artificial sequence
  Ribosome binding site
atcattacatagagggtatttaatt

SEQ ID NO: 16 - DNA, 25 bp, Artificial sequence
  Ribosome binding site
tatatttactagagggtatttaaaa

SEQ ID NO: 17 - DNA, 25 bp, Escherichia coli
catcgaaaacacggaggaagtatag

SEQ ID NO: 18 - DNA, 25 bp, Artificial sequence
  Ribosome binding site
taacatatactagagggtttaaata

SEQ ID NO: 19 - DNA, 25 bp, Artificial sequence
  Ribosome binding site
ttcatttcatagagggtatttaatt

SEQ ID NO: 20 - DNA, 25 bp, Artificial sequence
  Ribosome binding site
ctaaattgacagagaggataaaaga

SEQ ID NO: 21 - DNA, 25 bp, Escherichia coli
ttacatataattagaggaagaaaaa

SEQ ID NO: 22 - DNA, 25 bp, Artificial sequence
  Ribosome binding site
gtaaattactagagggtattaaaat

SEQ ID NO: 23 - DNA, 25 bp, Artificial sequence
  Ribosome binding site
gttaaaaactagagggtattaaata

SEQ ID NO: 24 - DNA, 25 bp, Artificial sequence
  Ribosome binding site
gtcacttaatagagggtatttaaaa

SEQ ID NO: 25 - DNA, 25 bp, Artificial sequence
  Ribosome binding site
gacactaaatagagggtatttaatt

SEQ ID NO: 26 - DNA, 69 bp, Artificial sequence
  Promoter sequence
tactgtatgagcatacagttaagggttgacaaccgatatttattcacttaatatataaatatcaactga

SEQ ID NO: 27 - DNA, 577 bp, Bos taurus
gccttcccagccatgtccttgtccggcctgtttgccaacgctgtgctccgggctcagcacctgcatcagctggctgctgacaccttcaaagagtttgagcgcacctacatcccggagggacagagatactccatccagaacacccaggttgccttctgcttctctgaaaccatcccggcccccacgggcaagaatgaggcccagcagaaatcagacttggagctgcttcgcatctcactgctcctcatccagtcgtggcttgggcccctgcagttcctcagcagagtcttcaccaacagcttggtgtttggcacctcggaccgtgtctatgagaagctgaaggacctggaggaaggcatcctggccctgatgcgggagctggaagatggcaccccccgggctgggcagatcctcaagcaggacctatgacaaatttgacacaaacatgcgcagtgacgacgcgctgctcaagaactacggtctgctctcctgcttccggaaggacctgcataagacggagacgtacctgagggtcatgaagtgccgccgcttcggggaggccagctgtgccttctag

SEQ ID NO: 28 - DNA, 977 bp, unknown
  Plasmid pXT709
gaattctactgtatgagcatacagttaagggttgacaaccgatatttattcacttaatatataaatatcaactgaggcgcgcccataacatcaagaggatatgaaattatgtttccagccatgagcttgtccggactctttgccaatgctgtactccgggctcagcacctgcatcagctggctgctgacaccttcaaagagtttgagcgcacctacatcccggagggacagcgttactccatccagaacacccaggttgccttctgcttctctgaaaccatcccggccccgacgggcaagaatgaggcccagcagaaatcagacttggagctgcttcgcatctcactgctcctcatccagagctggcttgggccgctgcagttcctcagccgtgtcttcaccaacagcttggtgtttggcaccagcgaccgtgtctatgagaagctgaaggacctggaggaaggcatcctggccctgatgcgtgagctggaagatggcaccccgcgtgctgggcagatcctcaagcagacctatgacaaatttgacacaaacatgcgcagtgacgacgcgctgctcaagaactacggtctgctctcctgcttccgtaaggacctgcataagacggagacgtacctgcgtgtcatgaagtgccgccgcttcggggaggcgagctgcgccttctaatagctcgagtctagactcactcattaggcaccccaggctttacactttatgcttccggctcgtataatgtgtggaattgtgagcggataacaatttcacacaggaaaaagctgattgcccttcaccgcctggcctccgttgagccatctggatcggcagcgttgtcttcatcaaccggaacgagcatgccggagagcagctcactcattaggcaccccaggctttacactttatgcttccggctcgtataatgtgtggaattgtgagcggataacaatttcacacagaagctt

SEQ ID NO: 29 - protein, 191 aa, Bos taurus
AFPAMSLSGLFANAVLRAQHLHQLAADTFKEFERTYIPEGQRYSIQNTQVAFCFSETIPAPTGKNEAQQKSDLELLRISLLLIQSWLGPLQFLSRVFTNSLVFGTSDRVYEKLKDLEEGILALMRELEDGTPRAGQILKQTYDKFDTNMRSDDALLKNYGLLSCFRKDLHKTETYLRVMKCRRFGEASCAF

SEQ ID NO: 30 - DNA, 25 bp, Escherichia coli
cataacatcaagaggatatgaaatt

SEQ ID NO: 31 - DNA, 25 bp, Escherichia coli
acaacctaacgatgaggtcttgatt

SEQ ID NO: 32 - DNA, 25 bp, Escherichia coli
cgcggccttaaaccgaggttttccc

SEQ ID NO: 33 - DNA, 25 bp, Artificial sequence
  Ribosome binding site
gtacaaatcatagagggtatttaat

SEQ ID NO: 34 - DNA, 25 bp, Escherichia coli
acatattaaatagtaggagtgcata

SEQ ID NO: 35 - DNA, 25 bp, Escherichia coli
ataaaataattggagctctggtctg

SEQ ID NO: 36 - DNA, 25 bp, Escherichia coli
aaaagctaaaaccaggagctattta

SEQ ID NO: 37 - DNA, 25 bp, Escherichia coli
attctatatactttggagttttaaa

SEQ ID NO: 38 - DNA, 25 bp, Escherichia coli
aaaaaaaccatgagggtaataaata

SEQ ID NO: 39 - DNA, 25 bp, Escherichia coli
ttaaatataaacctgaagattaaac

SEQ ID NO: 40 - DNA, 25 bp, Escherichia coli
aggcatataacagagggttaataac

SEQ ID NO: 41 - DNA, 25 bp, Artificial sequence
  Ribosome binding site
gctaaaatacggggaggtgataata

SEQ ID NO: 42 - DNA, 25 bp, Artificial sequence
  Ribosome binding site
gtacatactaggaggggttataata

SEQ ID NO: 43 - DNA, 25 bp, Escherichia coli
gagcactatagtaaggaatataggc

SEQ ID NO: 44 - DNA, 25 bp, Escherichia coli
tttggatgataacgaggcgcaaaaa

SEQ ID NO: 45 - DNA, 25 bp, Escherichia coli
gctgtgaaggcgtaaggagatagca

SEQ ID NO: 46 - DNA, 25 bp, Escherichia coli
caggttatttcgtagaggatttaag

SEQ ID NO: 47 - DNA, 25 bp, Artificial sequence
  Ribosome binding site
taaaagagactaggaggagattaga

SEQ ID NO: 48 - DNA, 25 bp, Escherichia coli
ggtgtgcgcggaagcggagttttat

SEQ ID NO: 49 - DNA, 25 bp, Escherichia coli
tgttttattcacacaagaggatgtt

SEQ ID NO: 50 - DNA, 25 bp, Escherichia coli
tatccgtgtcttagagggacaatcg

SEQ ID NO: 51 - DNA, 25 bp, Escherichia coli
aaccagaagaatggggattctcagg

SEQ ID NO: 52 - DNA, 25 bp, Escherichia coli
ccagtagcgctcgaaggagaggtga

SEQ ID NO: 53 - DNA, 25 bp, Escherichia coli
attaaaataaatgaaggattatgta

SEQ ID NO: 54 - DNA, 25 bp, Escherichia coli
gggcctggtagtggaggactaagaa

SEQ ID NO: 55 - DNA, 25 bp, Escherichia coli
attgagcgattgagaggttgaaaca

SEQ ID NO: 56 - DNA, 25 bp, Escherichia coli
tcactttatagttgaggatattacg

SEQ ID NO: 57 - DNA, 25 bp, Escherichia coli
gaaaacaatctaaggaatttttcaa

SEQ ID NO: 58 - DNA, 25 bp, Escherichia coli
taaacctataagttggggaaataca

SEQ ID NO: 59 - DNA, 25 bp, Escherichia coli
gttaactaaatagaggcattgtgct

SEQ ID NO: 60 - DNA, 25 bp, Escherichia coli
ggactttctcaaaggagagttatca

SEQ ID NO: 61 - DNA, 25 bp, Escherichia coli
gcggcatacctcgaagggagcagga

SEQ ID NO: 62 - DNA, 25 bp, Escherichia coli
tattacccaacttgaggaatttata

SEQ ID NO: 63 - DNA, 25 bp, Escherichia coli
ataacgactacaggacatcttgaat

SEQ ID NO: 64 - DNA, 25 bp, Escherichia coli
ctgcataagaaaaaccggagatacc

SEQ ID NO: 65 - DNA, 25 bp, Escherichia coli
aaaaagactgtcagttgaggacatt

SEQ ID NO: 66 - DNA, 25 bp, Escherichia coli
acatttgtttaaggaatgattaatt

SEQ ID NO: 67 - DNA, 25 bp, Escherichia coli
tattcaagtttaggtagaaatcgcc

SEQ ID NO: 68 - DNA, 25 bp, Escherichia coli
tcttaaacaattggaggaataaggt

SEQ ID NO: 69 - DNA, 25 bp, Escherichia coli
tatttgaatcacatttgaggtggtt

SEQ ID NO: 70 - DNA, 25 bp, Escherichia coli
ttataatgagggagtccaaaaaaca

SEQ ID NO: 71 - DNA, 25 bp, Escherichia coli
ctagctcaataaaagaaatcagacc

SEQ ID NO: 72 - DNA, 25 bp, Escherichia coli
caggattaatttaagagagaaagaa

SEQ ID NO: 73 - DNA, 25 bp, Escherichia coli
atagaacacatttgggagttggacc

SEQ ID NO: 74 - DNA, 25 bp, Escherichia coli
ctacgtagcctaatcggaggaaaaa

SEQ ID NO: 75 - DNA, 25 bp, Escherichia coli
ccacatatttttaaaggaaacagac

SEQ ID NO: 76 - DNA, 25 bp, Escherichia coli
caaacatcacaattggagcagaata

SEQ ID NO: 77 - DNA, 25 bp, Escherichia coli
aaaataaatcattgaggttatggtc

SEQ ID NO: 78 - DNA, 25 bp, Escherichia coli
gacttgcaatataggataacgaatc

SEQ ID NO: 79 - DNA, 25 bp, Escherichia coli
agacataacaacattcggggtgaat

SEQ ID NO: 80 - DNA, 25 bp, Escherichia coli
cggtagtgataccccagaggattag

SEQ ID NO: 81 - DNA, 25 bp, Escherichia coli
tggcaaacatccaggagcaaagcta

SEQ ID NO: 82 - DNA, 25 bp, Escherichia coli
taactatttattgggtaagctttta

SEQ ID NO: 83 - DNA, 25 bp, Escherichia coli
tagttgacattagcggagcactaaa

SEQ ID NO: 84 - DNA, 25 bp, Escherichia coli
ctaatttgatttttggagaatagac

SEQ ID NO: 85 - DNA, 25 bp, Escherichia coli
tgaataatccgtaaggagcaattcg

SEQ ID NO: 86 - DNA, 25 bp, Escherichia coli
cgaattaatcaaaggtgagaggcac

SEQ ID NO: 87 - DNA, 25 bp, Escherichia coli
gggattttcaaacagtggcatacat

SEQ ID NO: 88 - DNA, 11 bp, Artificial sequence
  LOAD sequence motif
tagagggtatt

SEQ ID NO: 89 - DNA, 25 bp, Escherichia coli
taactcaatctagagggtattaata

SEQ ID NO: 90 - DNA, 25 bp, Artificial sequence
  Ribosome binding site
ddwhahwwhmtagagggtattwaaw

SEQ ID NO: 91 - DNA, 25 bp, Artificial sequence
  Ribosome binding site
nnwmanwnwmnnrrrggwnnwwana

SEQ ID NO: 92 - DNA, 47 bp, Artificial sequence
  Double ribosome binding site
tctagagggtattaataatctatcgattaaataaggaggaataacat

SEQ ID NO: 93 - DNA, 25 bp, Escherichia coli
agttcacgtaaaaagggtatcgaca

SEQ ID NO: 94 - DNA, 25 bp, Artificial sequence
  Ribosome binding site
acgtaaaaagggtatctagaattct

SEQ ID NO: 95 - DNA, 38 bp, Artificial sequence
  Modified trpL ribosome binding site
aagttcacgttattaaaaattaaagaggtatatcgata

SEQ ID NO: 96 - DNA, 25 bp, Artificial sequence
  Ribosome binding site
ccatgtactaaggaggttcagatct

SEQ ID NO: 97 - DNA, 25 bp, Bacteriophage Lambda
ttgttatctaaggaagtacttacat

SEQ ID NO: 98 - DNA, 25 bp, Bacteriophage Mu
tacaaaacttaggagggtttttacc

SEQ ID NO: 99 - DNA, 25 bp, Artificial sequence
  Ribosome binding site
aaattagagaataacccggatccgg

SEQ ID NO: 100 - DNA, 25 bp, Artificial sequence
  Ribosome binding site
aaaattagagaataaccggatccgg

SEQ ID NO: 101 - DNA, 25 bp, Artificial sequence
  Ribosome binding site
caaaattagagaataccggatccgg

SEQ ID NO: 102 - DNA, 25 bp, Artificial sequence
  Ribosome binding site
acaaaattagagaatccggatccgg

SEQ ID NO: 103 - DNA, 25 bp, Artificial sequence
  Ribosome binding site
aacaaaattagagaaccggatccgg

SEQ ID NO: 104 - DNA, 25 bp, Artificial sequence
  Ribosome binding site
gaacaaaattagagaccggatccgg

SEQ ID NO: 105 - DNA, 25 bp, Artificial sequence
  Ribosome binding site
tgaacaaaattagagccggatccgg

SEQ ID NO: 106 - DNA, 25 bp, Artificial sequence
  Ribosome binding site
mhwhhwhdwhmdrgaggrwrtwwrm

SEQ ID NO: 107 - DNA, 576 bp, Bos taurus
gccttcccagccatgtccttgtccggcctgtttgccaacgctgtgctccgggctcagcacctgcatcagctggctgctgacaccttcaaagagtttgagcgcacctacatcccggagggacagagatactccatccagaacacccaggttgccttctgcttctctgaaaccatcccggcccccacgggcaagaatgaggcccagcagaaatcagacttggagctgcttcgcatctcactgctcctcatccagtcgtggcttgggcccctgcagttcctcagcagagtcttcaccaacagcttggtgtttggcacctcggaccgtgtctatgagaagctgaaggacctggaggaaggcatcctggccctgatgcgggagctggaagatggcaccccccgggctgggcagatcctcaagcagacctatgacaaatttgacacaaacatgcgcagtgacgacgcgctgctcaagaactacggtctgctctcctgcttccggaaggacctgcataagacggagacgtacctgagggtcatgaagtgccgccgcttcggggaggccagctgcgccttctag

SEQ ID NO: 108 - DNA, 576 bp, Bos taurus
gccttcccagccatgtccttgtccggcctgtttgccaacgctgtgctccgggctcagcacctgcatcagctggctgctgacaccttcaaagagtttgagcgcacctacatcccggagggacagagatactccatccagaacacccaggttgccttctgcttctctgaaaccatcccggcccccacgggcaagaatgaggcccagcagaaatcagacttggagctgcttcgcatctcactgctcctcatccagtcgtggcttgggcccctgcagttcctcagcagagtcttcaccaacagcttggtgtttggcacctcggaccgtgtctatgagaagctgaaggacctggaggaaggcatcctggccctgatgcgggaggtggaagatggcaccccccgggctgggcagatcctcaagcagacctatgacaaatttgacacaaacatgcgcagtgacgacgcgctgctcaagaactacggtctgctctcctgcttccggaaggacctgcataagacggagacgtacctgagggtcatgaagtgccgccgcttcggggaggccagctgtgccttctag

SEQ ID NO: 109 - DNA, 576 bp, Bos taurus
gccttcccagccatgtccttgtccggcctgtttgccaacgctgtgctccgggctcagcacctgcatcagctggctgctgacaccttcaaagagtttgagcgcacctacatcccggagggacagagatactccatccagaacacccaggttgccttctgcttctctgaaaccatcccggcccccacgggcaagaatgaggcccagcagaaatcagacttggagctgcttcgcatctcactgctcctcatccagtcgtggcttgggcccctgcagttcctcagcagagtcttcaccaacagcttggtgtttggcacctcggaccgtgtctatgagaagctgaaggacctggaggaaggcatcctggccctgatgcgggaggtggaagatggcaccccccgggctgggcagatcctcaagcagacctatgacaaatttgacacaaacatgcgcagtgacgacgcgctgctcaagaactacggtctgctctcctgcttccggaaggacctgcataagacggagacgtacctgagggtcatgaagtgccgccgcttcggggaggccagctgcgccttctag

SEQ ID NO: 110 - protein, 191 aa, Bos taurus
AFPAMSLSGLFANAVLRAQHLHQLAADTFKEFERTYIPEGQRYSIQNTQVAFCFSETIPAPTGKNEAQQKSDLELLRISLLLIQSWLGPLQFLSRVFTNSLVFGTSDRVYEKLKDLEEGILALMREVEDGTPRAGQILKQTYDKFDTNMRSDDALLKNYGLLSCFRKDLHKTETYLRVMKCRRFGEASCAF

SEQ ID NO: 111 - DNA, 26 bp, Artificial sequence
  Ribosome binding site
ddwhahwwhmtagagggtattwaaww

SEQ ID NO: 112 - protein, 191 aa, Bos taurus
MFPAMSLSGLFANAVLRAQHLHQLAADTFKEFERTYIPEGQRYSIQNTQVAFCFSETIPAPTGKNEAQQKSDLELLRISLLLIQSWLGPLQFLSRVFTNSLVFGTSDRVYEKLKDLEEGILALMRELEDGTPRAGQILKQTYDKFDTNMRSDDALLKNYGLLSCFRKDLHKTETYLRVMKCRRFGEASCAF

SEQ ID NO: 113 - protein, 191 aa, Bos taurus
MFPAMSLSGLFANAVLRAQHLHQLAADTFKEFERTYIPEGQRYSIQNTQVAFCFSETIPAPTGKNEAQQKSDLELLRISLLLIQSWLGPLQFLSRVFTNSLVFGTSDRVYEKLKDLEEGILALMREVEDGTPRAGQILKQTYDKFDTNMRSDDALLKNYGLLSCFRKDLHKTETYLRVMKCRRFGEASCAF